(19) United States
(12) Patent Application Publication
(10) Pub. No.: US 2026/0114816 A1
(43) Pub. Date: Apr. 30, 2026

(54) MEDICAL INFORMATION PROCESSING APPARATUS, SYSTEM, METHOD, AND STORAGE MEDIUM

(71) Applicant: CANON MEDICAL SYSTEMS CORPORATION, Tochigi (JP)

(72) Inventors: Seiji SUZUKI, Yokohama (JP); Yuto ITO, Kokubunji (JP); Junichi OOIDA, Ota-ku (JP); Isao KAWATA, Kawasaki (JP); Koshiro SUZUKI, Yokohama (JP); Yasuko FUJISAWA, Nasushiobara (JP); Atsuko SUGIYAMA, Ota-ku (JP); Kazuki UTSUNOMIYA, Nasushiobara (JP); Katsuhiko FUJIMOTO, Saitama (JP)

(73) Assignee: CANON MEDICAL SYSTEMS CORPORATION, Tochigi (JP)

(21) Appl. No.: 19/376,026

(22) Filed: Oct. 31, 2025

(30) Foreign Application Priority Data
Oct. 31, 2024 (JP) 2024-192434
Apr. 11, 2025 (JP) 2025-065920

(51) Int. Cl. A61B 5/00; G16H 20/10; G16H 30/20; G16H 30/40

(52) U.S. Cl. CPC A61B 5/7275; G16H 20/10; G16H 30/20; G16H 30/40

(57) ABSTRACT

A medical information processing apparatus according to an embodiment includes processing circuitry. The processing circuitry is configured to acquire subject information. The processing circuitry is configured to identify parameters of at least one or more physical models among a plurality of physical models included in a prediction model for predicting the behavior of the heart using the subject information.

### CROSS-REFERENCE TO RELATED APPLICATIONS

This application is based upon and claims the benefit of priority from Japanese Patent Application No. 2024-192434, filed on Oct. 31, 2024; and Japanese Patent Application No. 2025-065920, filed on Apr. 11, 2025, the entire contents of all of which are incorporated herein by reference.

### FIELD

Embodiments described herein relate generally to a medical information processing apparatus, a system, a method, and a storage medium.

### BACKGROUND

In recent years, with the fragmentation and complication of clinical judgment, the burden on doctors has increased, and expectations for clinical decision support (CDS) for reducing such a burden have increased. For such support, application of physical simulation has entered a practical stage, and for example, prediction of fractional flow reserve (FFR) after treatment for the coronary artery, postoperative prediction of transcatheter aortic valve implantation (TAVI), and the like are performed by physical simulation.

In medication treatment for the heart, in order to evaluate the effect of medication, measurement of a change in membrane potential by a patch clamp method using collected cardiomyocytes, blood examination after administration, observation of the state of blood flow after administration, and the like are performed. Therefore, in order to reduce the burden on the patient and the doctor regarding the medication treatment, it is required to predict the pharmacological effect at the stage of the medication plan before the medication.

### BRIEF DESCRIPTION OF THE DRAWINGS

FIG. 1 is a block diagram illustrating an example of a configuration of a medical information processing system according to a first embodiment;

FIG. 2 is a flowchart illustrating an example of a processing procedure of a medical information processing apparatus according to the first embodiment;

FIG. 3 is a diagram illustrating an example of a prediction model according to the first embodiment;

FIG. 4 is a diagram illustrating an example of parameter identification processing according to the first embodiment;

FIG. 5A is a diagram illustrating a pharmacological effect prediction framework (workflow for pharmacological effect prediction) using a personalized hierarchical approach;

FIG. 5B is a diagram illustrating a pharmacological effect prediction framework (overview of hierarchical personalization) using a personalized hierarchical approach;

FIG. 6 is a diagram illustrating a cylindrical model of the left ventricle (A cylindrical model assuming an LV equator region was considered, and three-layer modeling was adopted for electrochemical and fiber directions);

FIG. 7A is a diagram illustrating settings of structural analysis (load condition and displacement constraint in a cylindrical cross section) for a cylindrical model;

FIG. 7B is a diagram illustrating settings of structural analysis (mesh of the cylindrical model) for the cylindrical model;

FIG. 8 is a diagram illustrating an algorithm of parameter optimization for the ORd model and the deformation model;

FIG. 9 is a diagram illustrating operation verification of the ORd model (Calculated from Endo parameter values described in FIG. 10 of literature);

FIG. 10 is a diagram illustrating operation verification of the Land model (The time evolution of the contraction force in the isometric condition described in FIG. 6 of literature [7] was compared);

FIG. 11 is a diagram illustrating operation verification of the ventricular deformation model (comparison of the relationship between the internal pressure and the inner diameter of the cylindrical model);

FIG. 12 is a diagram illustrating a history of loss functions in virtual personalization of the ORd model;

FIG. 13 is a diagram illustrating the reproducibility of the action potential in the virtual personalization of the ORd model;

FIG. 14A is a diagram illustrating a virtual personalization result (time history of input virtual distortion) of the Land model;

FIG. 14B is a diagram illustrating a virtual personalization result (a calculation result of Tₐ) of the Land model;

FIG. 15A is a diagram illustrating a virtual personalization result (comparison between an input virtual patient P-V loop and a calculation result) of the deformation model;

FIG. 15B is a diagram illustrating a virtual personalization result (loss function transition) of the deformation model;

FIG. 16 is a diagram illustrating an outline of verification regarding parameter indefiniteness of hierarchical personalization;

FIG. 17A is a diagram illustrating the influence (action potential) of different personalizations on the ORd model calculation result (Calculation was performed by setting the initial average value of optimization to magnifications of 0.7, 0.9, 1.0, 1.1, and 1.3 with respect to the reference value);

FIG. 17B is a diagram illustrating the influence (transition of loss function in the backcast approach) of different personalizations on the ORd model calculation result (Calculation was performed by setting the initial average value of optimization to magnifications of 0.7, 0.9, 1.0, 1.1, and 1.3 with respect to the reference value);

FIG. 18A is a diagram illustrating correlations of optimized model parameters (Two parameter sets optimized for action potential or calcium ion distribution were plotted for each ratio of the initial value to the reference value);

FIG. 18B is a diagram illustrating correlations of optimized model parameters (Two parameter sets optimized for action potential or calcium ion distribution were plotted for each ratio of the initial value to the reference value);

FIG. 18C is a diagram illustrating correlations of optimized model parameters (Two parameter sets optimized for action potential or calcium ion distribution were plotted for each ratio of the initial value to the reference value);

FIG. 18D is a diagram illustrating correlations of optimized model parameters (Two parameter sets optimized for action potential or calcium ion distribution were plotted for each ratio of the initial value to the reference value);

FIG. 18E is a diagram illustrating correlations of optimized model parameters (Two parameter sets optimized for action potential or calcium ion distribution were plotted for each ratio of the initial value to the reference value);

FIG. 19A is a diagram illustrating a comparison of electrochemical and contraction kinetics of cells in the Endo layer before and after administration;

FIG. 19B is a diagram illustrating a comparison of electrochemical and contraction kinetics of cells in the Endo layer before and after administration;

FIG. 19C is a diagram illustrating a comparison of electrochemical and contraction kinetics of cells in the Endo layer before and after administration; and

FIG. 20 is a diagram illustrating a comparison of P-V loops before and after administration.

### DETAILED DESCRIPTION

A medical information processing apparatus according to an embodiment includes processing circuitry. The processing circuitry is configured to acquire subject information. The processing circuitry is configured to identify parameters of at least one or more physical models among a plurality of physical models included in a prediction model for predicting the behavior of the heart using the subject information.

Hereinafter, embodiments of a medical information processing apparatus, a system, a method, and a program will be described in detail with reference to the drawings. Note that the medical information processing apparatus, system, method, and program according to the present application are not limited to the following embodiments.

### First Embodiment

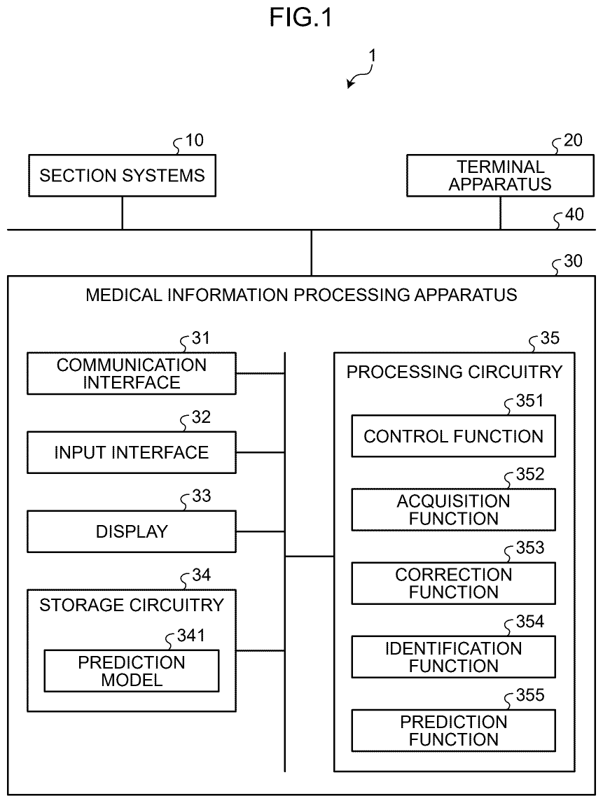

Hereinafter, a configuration of a medical information processing apparatus according to a first embodiment will be described with reference to FIG. 1. FIG. 1 is a block diagram illustrating an example of a configuration of a medical information processing system according to the first embodiment. For example, as illustrated in FIG. 1, a medical information processing system 1 includes section systems 10, a terminal apparatus 20, and a medical information processing apparatus 30. Section systems 10, the terminal apparatus 20, and the medical information processing apparatus 30 are communicably connected to each other via a network 40. Here, the network includes, for example, an in-hospital local area network (LAN) or a wide area network (WAN) installed in a hospital.

Note that various other apparatuses and systems such as a medical image diagnostic apparatus may be connected to the network illustrated in FIG. 1. The medical image diagnostic apparatus is an apparatus that captures an image of a subject to generate a medical image, and includes, for example, an X-ray computed tomography (CT) apparatus, a magnetic resonance imaging (MRI) apparatus, an X-ray diagnostic apparatus, an ultrasonic diagnostic apparatus, a single photon emission computed tomography (SPECT) apparatus, a positron emission computed tomography (PET) apparatus, and the like.

Section systems 10 includes various systems such as a hospital information system (HIS), a radiology information system (RIS), a picture archiving and communication system (PACS), a diagnosis report system, a laboratory information system (LIS), a rehabilitation section system, a dialysis section system, and a surgery section system. Section systems 10 manages subject information (patient information) for each subject (patient), and transmits the subject information in response to requests from the terminal apparatus 20 and the medical information processing apparatus 30. Here, the subject information (patient information) includes, for example, various medical images collected by the medical image diagnostic apparatus, a measurement value measured using the medical image, attribute information (sex and the like), a measured value (height, weight, blood pressure, and the like), presence and/or absence of medication, and the like, and is managed in association with information (date and time of collection, storage location of data, and the like) regarding data of the subject information.

The terminal apparatus 20 is an apparatus operated by a doctor working in a hospital or the like. For example, the terminal apparatus 20 is implemented by a personal computer, a tablet PC, a PDA, a mobile phone such as a smartphone, or the like. The terminal apparatus 20 displays various types of information on a display of the terminal apparatus and receives various operations via an input interface of the terminal apparatus. Here, the terminal apparatus 20 can transmit a processing request to the medical information processing apparatus 30 and receive a processing result in response to an operation of a doctor or the like.

As illustrated in FIG. 1, the medical information processing apparatus 30 includes a communication interface 31, an input interface 32, a display 33, storage circuitry 34, and processing circuitry 35. For example, the medical information processing apparatus 30 is implemented by a computer device such as a server or a workstation.

The communication interface 31 controls transmission and communication of various data transmitted and received between the medical information processing apparatus 30 and each apparatus connected thereto via the network 40. Specifically, the communication interface 31 is connected to the processing circuitry 35, and transmits data received from each apparatus on the network 40 to the processing circuitry 35 or transmits data received from the processing circuitry 35 to each apparatus on the network 40. For example, the communication interface 31 is implemented by a network card, a network adapter, a network interface controller (NIC), or the like.

The input interface 32 receives various instructions and input operations of various types of information from an operator. Specifically, the input interface 32 is connected to the processing circuitry 35, converts the input operation received from the operator into an electric signal, and transmits the electric signal to the processing circuitry 35. For example, the input interface 32 is implemented by a trackball, a switch button, a mouse, a keyboard, a touch pad that performs an input operation by touching an operation surface, a touch screen in which a display screen and a touch pad are integrated, a non-contact input interface using an optical sensor, a voice input interface, and the like. Note that, in the present specification, the input interface 32 is not limited to one including physical operation components such as a mouse and a keyboard. For example, an electric signal processing circuit that receives an electric signal corresponding to an input operation from an external input device provided separately from the device and transmits the electric signal to the control circuit is also included in the example of the input interface 32.

The display 33 displays various types of information and various types of data. Specifically, the display 33 is connected to the processing circuitry 35 and displays various types of information and various types of data received from the processing circuitry 35. For example, the display 33 is implemented by a liquid crystal display, a cathode ray tube (CRT) display, a touch panel, or the like.

The storage circuitry 34 stores various data and various programs. Specifically, the storage circuitry 34 is connected to the processing circuitry 35, stores the data received from the processing circuitry 35, or reads the stored data and transmits the data to the processing circuitry 35. For example, the storage circuitry 34 is implemented by a semiconductor memory element such as a read only memory (ROM), a random-access memory (RAM), or a flash memory, a hard disk, an optical disk, or the like. For example, the storage circuitry 34 stores subject information received from section systems 10 and various programs. In addition, the storage circuitry 34 stores a prediction model 341 as illustrated in FIG. 1. The prediction model 341 is a simulation model that outputs a pharmacological effect according to an input of drug information (for example, the molecular structure of a drug), and details thereof will be described later. Note that the storage circuitry 34 may be implemented by a cloud computer connected to the medical information processing apparatus 30 via the network 40.

The processing circuitry 35 controls the entire medical information processing apparatus 30. Specifically, the processing circuitry 35 controls transmission and reception of information with each apparatus on the network 40 to control various types of processing related to prediction of a pharmacological effect. Note that the processing circuitry 35 can also perform various types of processing in response to an input operation via the input interface 32.

The configuration example of the medical information processing apparatus 30 according to the present embodiment has been described above. For example, the medical information processing apparatus 30 is installed in a medical facility such as a hospital, and supports prediction of a pharmacological effect of a drug for the heart performed by a user such as a doctor.

In a physical simulation for predicting the behavior of the heart, there is known a technique for selecting parameters for each patient by limiting parameters of a model included in the simulation, creating a database of simulation results performed by swinging levels of the parameters, and comparing a heart shape or an echo result of the patient (patient data) with a simulation result of the database. However, in the above technique, in a case where there is a plurality of parameter sets in which similar results are obtained as simulation results, even if patient data and a database are compared, one parameter set cannot be selected, and patient reproducibility is not necessarily high in some cases.

Therefore, the medical information processing apparatus 30 according to the present embodiment identifies the parameters of the plurality of physical models constituting the prediction model for predicting the behavior of the heart using the patient information (subject information), thereby improving the patient reproducibility of the simulation and accurately predicting the pharmacological effect for each patient. Hereinafter, the medical information processing apparatus 30 having such a configuration will be described in detail.

For example, as illustrated in FIG. 1, the processing circuitry 35 of the medical information processing apparatus 30 executes a control function 351, an acquisition function 352, a correction function 353, an identification function 354, and a prediction function 355. Here, the acquisition function 352 is an example of an acquisition unit. In addition, the correction function 353 is an example of a correction unit. In addition, the identification function 354 is an example of an identification unit.

The control function 351 controls various processes based on various requests input from the operator via the input interface 32 and various programs and various data read from the storage circuitry 34. For example, the control function 351 causes the display 33 to display a graphical user interface (GUI) for receiving an input related to prediction of a pharmacological effect, a prediction result, and the like.

The acquisition function 352 acquires subject information. Specifically, the acquisition function 352 acquires the subject information of the subject input via the input interface 32 from section systems 10. For example, the acquisition function 352 acquires medical images collected in a plurality of time phases for the heart of the subject and medical information of the subject. As an example, the acquisition function 352 acquires medical images collected for the ventricle of the heart of the subject, and medical information including the sex, blood pressure, height, and weight of the subject. That is, the acquisition function 352 acquires a medical image, a measurement value measured using the medical image, attribute information (sex and the like), a measured value (height, weight, blood pressure, and the like), and the like.

The correction function 353 corrects a numerical value based on the subject information acquired by the acquisition function 352. Specifically, the correction function 353 corrects the numerical value based on the subject information according to the state of the subject when the subject information is obtained from the subject. For example, the correction function 353 corrects measurement values measured using the medical image and measured values according to the state of the subject when the measurement values and the measured values are obtained. Note that details of processing by the correction function 353 will be described later.

The identification function 354 identifies parameters of at least one or more physical models among a plurality of physical models included in a prediction model for predicting the behavior of the heart using the subject information. Specifically, the identification function 354 identifies the parameters of each physical model using the subject information for each of the plurality of physical models constituting the prediction model 341 stored in the storage circuitry 34. For example, the identification function 354 identifies the parameters of each physical model by inverse analysis using the subject information. Note that details of processing by the identification function 354 will be described later.

The prediction function 355 predicts a pharmacological effect in the subject using the prediction model 341 (personalized prediction model 341) to which the parameter identified by the identification function 354 is applied. Specifically, the prediction function 355 predicts a pharmacological effect by inputting drug information (for example, the molecular structure of a drug) input from an operator via the input interface 32 to the personalized prediction model 341. Note that details of processing by the prediction function 355 will be described later.

The processing circuitry 35 described above is implemented by, for example, a processor. In that case, each processing function described above is stored in the storage circuitry 34 in the form of a program executable by a computer. Then, the processing circuitry 35 reads and executes each program stored in the storage circuitry 34 to implement a function corresponding to each program. In other words, the processing circuitry 35 has each processing function illustrated in FIG. 1 in a state where each program is read.

Note that the processing circuitry 35 may be configured by combining a plurality of independent processors, and each processor may implement each processing function by executing a program. Furthermore, each processing function of the processing circuitry 35 may be implemented by being appropriately distributed or integrated into a single or a plurality of processing circuits. In addition, each processing function of the processing circuitry 35 may be implemented by mixing hardware such as a circuit and software. Furthermore, here, an example of a case where the program corresponding to each processing function is stored in the single storage circuitry 34 has been described, but the embodiments are not limited thereto. For example, a plurality of storage circuits may dispersedly store programs corresponding to each processing function, and the processing circuitry 35 may read and execute each program from each storage circuit. Note that a part of each processing function included in the processing circuitry 35 may be implemented by a cloud computer connected to the medical information processing apparatus 30 via the network 40.

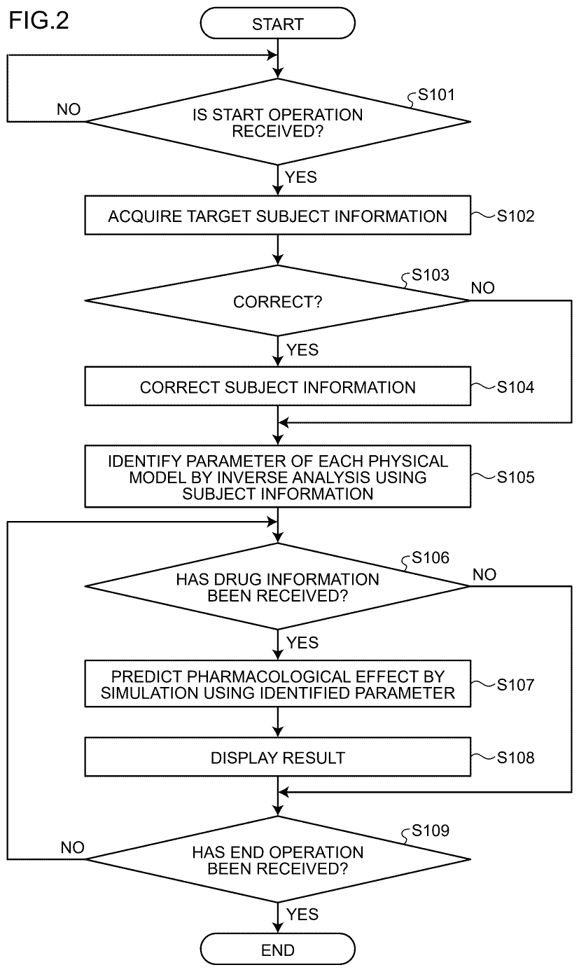

Next, a procedure of processing by the medical information processing apparatus 30 according to the first embodiment will be described with reference to FIG. 2, and then details of each processing will be described. FIG. 2 is a flowchart illustrating an example of a processing procedure of the medical information processing apparatus 30 according to the first embodiment.

For example, as illustrated in FIG. 2, in the medical information processing apparatus 30 according to the present embodiment, when a start operation is received (Step S101, Yes), the acquisition function 352 acquires subject information of a target subject (step S102). Note that the medical information processing apparatus 30 is in a standby state until receiving the start operation (Step S101, No). This processing is achieved, for example, by the processing circuitry 35 calling and executing a program corresponding to the acquisition function 352 from the storage circuitry 34.

Subsequently, the correction function 353 determines whether or not to correct the acquired subject information (step S103). Here, in a case where it is determined not to correct the subject information (Step S103, No), correction by the correction function 353 is not performed, and the processing proceeds to step S105. On the other hand, in a case where it is determined to correct the subject information, the correction function 353 corrects the subject information acquired by the acquisition function 352 by a correction method according to the state of the subject (step S104). This processing is achieved, for example, by the processing circuitry 35 calling and executing a program corresponding to the correction function 353 from the storage circuitry 34.

Subsequently, the identification function 354 identifies parameters of each physical model constituting the prediction model 341 by inverse analysis using the subject information acquired by the acquisition function 352 (alternatively, the subject information after correction by the correction function 353) (step S105). This processing is achieved, for example, by the processing circuitry 35 calling and executing a program corresponding to the identification function 354 from the storage circuitry 34.

Subsequently, the prediction function 355 determines whether drug information has been received (step S106). Here, in a case where drug information has been received (Step S106, Yes), the prediction function 355 predicts a pharmacological effect by simulation using the identified parameter (step S107), and the control function 351 displays a prediction result on the display 33 (step S108). This processing is achieved, for example, by the processing circuitry 35 calling and executing a program corresponding to the prediction function 355 and the control function 351 from the storage circuitry 34.

After the prediction result is displayed on the display 33 or when the drug information has not been received (Step S106, No), the prediction function 355 determines whether an end operation has been received (step S109). Here, in a case where the end operation has not been received (Step S109, No), the prediction function 355 continues the determination processing of step S106. On the other hand, in a case where the end operation has been received (Step S109, Yes), the medical information processing apparatus 30 ends the processing.

Hereinafter, details of each processing executed by the medical information processing apparatus 30 will be described.

### Processing of Acquiring Subject Information

As described in steps S101 and S102 of FIG. 2, the acquisition function 352 acquires the subject information when receiving the processing start operation. Specifically, the acquisition function 352 acquires target subject information from section systems 10 based on information for identifying a subject (for example, patient ID, name, and the like) input via the input interface 32. For example, the acquisition function 352 acquires medical information associated with the input patient ID, a medical image collected for the heart, a measurement value measured based on the medical image, and the like.

As an example, the acquisition function 352 acquires sex, DNA information, a measurement value (height, weight, blood pressure (cuff pressure), electrocardiogram, and the like) of the target subject, a morphological image of a plurality of time phases indicating a temporal change in shape of the heart, a result of myocardial strain analysis obtained by analyzing a contraction state of the myocardium, and the like. Here, the morphological image and the result of myocardial strain analysis acquired by the acquisition function 352 may be the entire heart or may be local, such as one cross section of the left ventricle.

The acquisition function 352 can also extract the shape of the heart from each of the morphological images of the plurality of time phases and acquire the deformation behavior of the heart from the extracted shape of each time phase. The acquisition function 352 can also acquire a result by performing myocardial strain analysis based on a medical image. Note that the subject information described above may be acquired by being simultaneously input at the time of inputting the subject information via the input interface 32.

### Processing of Correcting Subject Information

As described in steps S103 and S104 in FIG. 2, the correction function 353 determines whether to correct the subject information acquired by the acquisition function 352 and performs correction. In the subject information acquired by the acquisition function 352, the acquired date and the state of the subject may be different. Therefore, the correction function 353 corrects the subject information used for parameter identification according to the obtained state of the subject.

For example, the correction function 353 compares the latest blood pressure value acquired by the acquisition function 352 with the average value of the past blood pressures, and corrects the value of the blood pressure used to identify the parameter to an average value including the latest blood pressure value when there is a difference equal to or more than a threshold value. Furthermore, for example, in a case where the posture of the subject at the time of collection is different in the medical images collected at different times, the correction function 353 corrects the subject information so as to match one posture. In such a case, for example, the storage circuitry 34 stores in advance the tendency of the measurement value measured from the medical image collected in each posture, and the correction function 353 corrects the subject information based on the tendency.

In addition, for example, in a case where the subject information acquired by the acquisition function 352 when predicting the pharmacological effect of the second dose in the treatment of performing a plurality of doses is information of the date before the first dose, the correction function 353 corrects the acquired subject information to empirical information according to the treatment state. That is, the correction function 353 corrects the numerical value of the acquired subject information by the effect obtained by the first dose. In such a case, for example, the storage circuitry 34 stores in advance an average effect (improvement rate or the like) obtained for each drug, and the correction function 353 reads the average effect of the target drug and corrects the numerical value of the subject information.

### Parameter Identification Processing

As described in step S105 of FIG. 2, the identification function 354 identifies the parameter of each physical model included in the prediction model 341 using the subject information. Here, the prediction model 341 will be described. The prediction model 341 is a simulation model that outputs a pharmacological effect in response to an input of drug information (for example, the molecular structure of a drug), and is configured by combining a plurality of physical models. Specifically, the prediction model 341 includes at least one physical model among a molecular dynamic model that predicts an interaction between an ion channel on a cell membrane of the heart and a drug, an electrophysiological model that predicts at least one of an electrical change and a chemical change in cells of the heart, and a cross-bridge motion model that predicts a contraction of a sarcomere in a myocardium of the heart, and a cardiac deformation model that predicts a heartbeat state of the heart based on at least one prediction result.

For example, the prediction model 341 includes a molecular dynamic model, an electrophysiological model, a cross-bridge motion model, and a cardiac deformation model. The electrophysiological model predicts at least one (typically both) of an electrical change and a chemical change based on the prediction result of the molecular dynamic model. The cross-bridge motion model predicts the contraction of the sarcomere based on the prediction result of the electrophysiological model. The cardiac deformation model predicts a heartbeat state of the heart based on a prediction result of the cross-bridge motion model. The prediction model 341 may further include a blood flow model that predicts blood flow of the subject based on a prediction result of the cardiac deformation model.

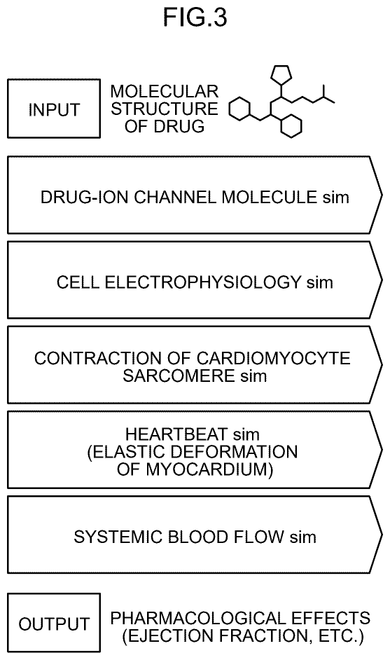

FIG. 3 is a diagram illustrating an example of a prediction model according to the first embodiment. For example, as illustrated in FIG. 3, the prediction model 341 includes a drug-ion channel molecular simulation (molecular dynamic model), an electrophysiological simulation of a cell (electrophysiological model), a contraction simulation of a cardiomyocyte sarcomere (cross-bridge motion model), a heartbeat simulation (elastic deformation of the myocardium) (cardiac deformation model), and a systemic blood flow simulation (blood flow model), and outputs a pharmacological effect (ejection fraction or the like) in response to an input of a molecular structure of a drug.

Here, the prediction model 341 has a hierarchical structure illustrated in FIG. 3. That is, the plurality of physical models included in the prediction model 341 includes a first physical model and a second physical model having an output of the first physical model as at least a part of an input. For example, in the prediction model 341 illustrated in FIG. 3, the output of the drug-ion channel molecular simulation is an input of the electrophysiological simulation of the cell, the output of the electrophysiological simulation of the cell is an input of the contraction simulation of the cardiomyocyte sarcomere, the output of the contraction simulation of the cardiomyocyte sarcomere is an input of the heartbeat simulation, and the output of the heartbeat simulation is an input of the systemic blood flow simulation.

The drug-ion channel molecular simulations predict the interaction of the drug with ion channels (sodium channel, potassium channel, calcium channel, and the like) on the cell membrane of the myocardium. Specifically, the drug-ion channel molecular simulation predicts the affinity (ease of uptake) of the input drug for the ion channel. The drug-ion channel molecular simulation is achieved by, for example, a physical model based on the MP-CAFEE method, the gREST method, the free energy perturbation method, the MM/PBSA method, or the like.

The electrophysiological simulation of cells predicts the behavior of the intracellular potential of the myocardium. Specifically, the electrophysiological simulation of a cell predicts a change in ion concentration in the cell due to a drug taken into the cell, and predicts a change in membrane potential of a cardiomyocyte membrane due to the change in ion concentration. The electrophysiological simulation of cells is achieved by, for example, an ORd model, a Tomek model, a Passini model, a BPS model, or the like.

The contraction simulation of cardiomyocyte sarcomere predicts a contraction of a sarcomere composed of actin filaments and myosin filaments. Specifically, the contraction simulation of cardiomyocyte sarcomere predicts a change in sarcomeric length from a change in calcium ion concentration in cardiomyocytes. The contraction simulation of the cardiomyocyte sarcomere is achieved by, for example, a Campbell model, a Land model, a phenomenological model using a Tanh function, a molecular dynamic model, or the like.

The heartbeat simulation predicts elastic deformation of the myocardium. Specifically, the heartbeat simulation predicts the change in the shape of the heart from the change in the sarcomeric length and the viscoelastic characteristics of the myocardial tissue. Here, the heartbeat simulation (cardiac deformation model) may be a simulation (deformation model) that predicts deformation in a cross section of the left ventricle of the heart. In this case, the deformation model may predict deformation in a longitudinal section or a transverse section of the left ventricle of the heart. The heartbeat simulation is achieved by, for example, a local sliced model, a cylindrical approximated left ventricular model, an elliptical approximated left ventricular model, or the like.

The systemic blood flow simulation predicts ejection of blood from the heart. For example, the systemic blood flow simulation predicts blood flow ejected from the heart based on changes in the shape of the heart and predicts ejection fraction. The systemic blood flow simulation is achieved by, for example, a Windkessel model, a circuit model, a one-dimensional fluid model, or the like.

Note that the prediction model 341 can output not only the final output from the systemic blood flow simulation but also the prediction result of each simulation.

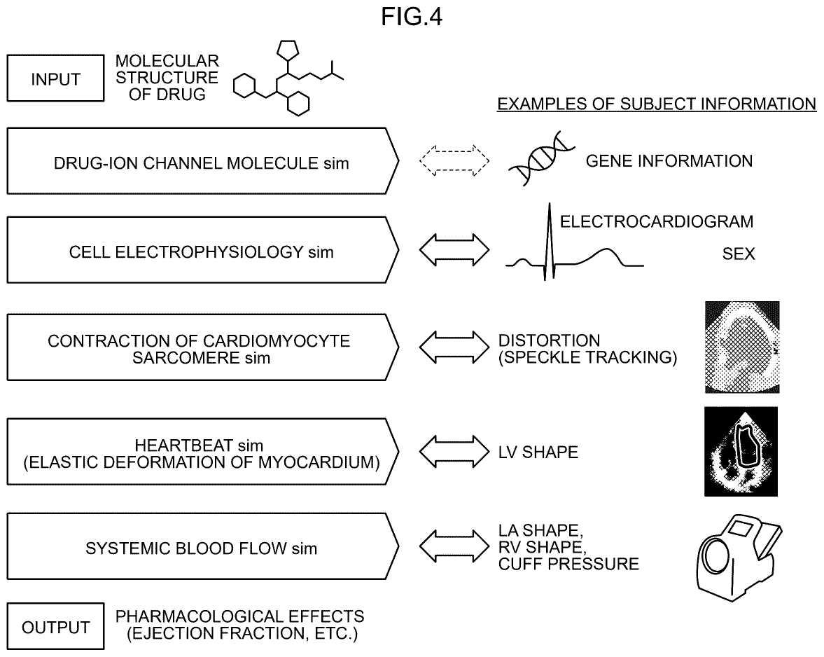

The identification function 354 identifies parameters by inverse analysis using the subject information for each simulation (each physical model) constituting the prediction model 341 illustrated in FIG. 3. That is, the identification function 354 individually identifies parameters for each simulation (each physical model) constituting the prediction model 341. FIG. 4 is a diagram illustrating an example of parameter identification processing according to the first embodiment.

For example, as illustrated in FIG. 4, the identification function 354 uses gene information to identify parameters of a drug-ion channel molecular simulation. Specifically, the identification function 354 acquires gene information of an ion channel in cardiomyocytes from DNA information acquired as subject information, and applies the gene information to drug-ion channel molecular simulation. That is, the identification function 354 identifies the parameter so that the structure of the protein constituting the ion channel matches the subject. This allows the drug-ion channel molecular simulation to make optimized (personalized) predictions for the target subject.

In addition, for example, as illustrated in FIG. 4, the identification function 354 identifies the parameters of the electrophysiological simulation of the cell using the electrocardiogram and the sex information. Specifically, the identification function 354 optimizes the parameters of the electrophysiological simulation of the cell such that the electrical change predicted by the electrophysiological simulation of the cell approximates the electrical signal indicated by the electrocardiogram. Here, since the waveform of the electrocardiogram (characteristics of the electrical stimulation of the cell) differs depending on the sex, the identification function 354 can also change the parameters of the electrophysiological simulation of the cell based on the sex of the subject. Thereby, the electrophysiological simulation of the cell can make a personalized prediction for the target subject.

In addition, for example, as illustrated in FIG. 4, the identification function 354 identifies parameters of a contraction simulation of cardiomyocyte sarcomere using an analysis result of myocardial strain by a speckle tracking method. Specifically, the identification function 354 optimizes the parameters of the contraction simulation of the cardiomyocyte sarcomere such that the change in the sarcomeric length predicted by the contraction simulation of the cardiomyocyte sarcomere corresponds to the contraction of the myocardium obtained by the strain analysis. As a result, the contraction simulation of the cardiomyocyte sarcomere can perform prediction personalized to the target subject.

Furthermore, for example, as illustrated in FIG. 4, the identification function 354 identifies the parameters of the heartbeat simulation using the shape change of the left ventricle (LV) measured in a plurality of medical images collected over time. Specifically, the identification function 354 optimizes the parameters of the heartbeat simulation such that the prediction result of the heartbeat simulation for predicting the elastic deformation of the left ventricle approximates the shape change of the left ventricle based on the medical image. Thus, the heartbeat simulation can make a personalized prediction for the target subject.

In addition, for example, as illustrated in FIG. 4, the identification function 354 identifies the parameters of the systemic blood flow simulation using the shape change of the left atrium (LA), the shape change of the right ventricle (RV), and the blood pressure (cuff pressure) measured by a plurality of medical images collected over time. Specifically, the identification function 354 optimizes the parameters of the systemic blood flow simulation such that the prediction result of the systemic blood flow simulation for predicting the blood flow becomes the blood flow corresponding to the shape change of the left atrium and the right ventricle and the cuff pressure based on the medical image.

Systemic blood flow varies depending on the body type of the subject. Therefore, for example, the identification function 354 can identify the parameter so that the blood flow rate increases when the body shape is large, and can identify the parameter so that the blood flow rate decreases when the body shape is small. Thus, the systemic blood flow simulation can make a personalized prediction for the target subject.

When the parameter of each physical model is identified as described above, the identification function 354 stores the parameter of each physical model in the storage circuitry 34 in association with the subject information. Note that the parameters of each physical model may be managed by section systems 10.

### Prediction Processing of Pharmacological Effect

As described in steps S106 and S107 in FIG. 2, when drug information is received, the prediction function 355 predicts a pharmacological effect of the received drug using the prediction model 341 to which the identified parameter for each physical model is applied. Specifically, the prediction function 355 inputs the drug information (for example, the molecular structure of a drug) input via the input interface 32 to the prediction model 341 personalized to each physical model, thereby predicting the blood flow (for example, ejection fraction) ejected from the heart. The prediction function 355 stores the prediction result in the storage circuitry 34 in association with the subject information. Note that the prediction result may be managed by section systems 10.

### Prediction Result Display Processing

As described in step S108 of FIG. 2, the control function 351 causes the display 33 to display the prediction result by the prediction function 355. For example, the control function 351 causes the display 33 to display the ejection fraction predicted by the prediction function 355. Here, the control function 351 can also display the latest ejection fraction on the display 33 together with the ejection fraction before medication and the ejection fraction predicted in the past. Furthermore, the control function 351 can also display a prediction result by each physical model on the display.

The medical information processing apparatus 30 according to the present embodiment can be used not only in a case where a pharmacological effect is predicted before a medication treatment is started, but also in a case where a pharmacological effect is predicted before each medication in a treatment in which medication is performed a plurality of times.

As described above, according to the first embodiment, the acquisition function 352 acquires subject information. The identification function 354 identifies parameters of at least one or more physical models among a plurality of physical models included in a prediction model for predicting the behavior of the heart using the subject information. Therefore, the medical information processing apparatus 30 according to the first embodiment can construct a prediction model that reproduces the state of the subject by correcting the parameters of each physical model constituting the prediction model to those adapted to the subject (patient), and can accurately predict the pharmacological effect for each patient.

Since the simulation that analyzes the behavior of the heart with respect to the input of the drug is a combination of various physical models in a wide range from micro to macro, the number of parameters is large, and it is difficult to collectively select them. However, in the medical information processing apparatus 30 according to the present embodiment, it is possible to efficiently identify the parameter by identifying the parameter of each physical model constituting the prediction model for each hierarchy. In addition, by identifying the parameters for each hierarchy, it is possible to improve patient reproducibility in the simulation.

In addition, according to the first embodiment, the plurality of physical models includes a first physical model and a second physical model having an output of the first physical model as at least a part of an input. Therefore, the medical information processing apparatus 30 according to the first embodiment can improve the accuracy of prediction of the pharmacological effect for each patient by the prediction model in which a plurality of physical models are correlated with each other.

In addition, according to the first embodiment, the prediction model includes at least one physical model among a molecular dynamic model that predicts an interaction between an ion channel on a cell membrane of the heart and a drug, an electrophysiological model that predicts at least one of an electrical change and a chemical change in cells of the heart, and a cross-bridge motion model that predicts a contraction of a sarcomere in a myocardium of the heart, and a cardiac deformation model that predicts a heartbeat state of the heart based on at least one prediction result. Therefore, the medical information processing apparatus 30 according to the first embodiment can construct a prediction model combining various physical models in a wide range from micro to macro.

Furthermore, according to the first embodiment, the prediction model includes a molecular dynamic model, an electrophysiological model, a cross-bridge motion model, and a cardiac deformation model, the electrophysiological model predicts at least one of an electrical change and a chemical change based on a prediction result of the molecular dynamic model, the cross-bridge motion model predicts a contraction of a sarcomere based on a prediction result of the electrophysiological model, and the cardiac deformation model predicts a heartbeat state of the heart based on a prediction result of the cross-bridge motion model. Therefore, the medical information processing apparatus 30 according to the first embodiment can construct a prediction model that predicts the behavior of the heart for each hierarchy.

In addition, according to the first embodiment, the cardiac deformation model is a deformation model that predicts deformation in a cross section of the left ventricle of the heart. In addition, the deformation model predicts deformation in a longitudinal section or a transverse section of the left ventricle of the heart. Therefore, the medical information processing apparatus 30 according to the first embodiment can reduce the calculation cost by predicting the local deformation of the heart.

In addition, according to the first embodiment, the prediction model further includes a blood flow model that predicts blood flow of the subject based on a prediction result of the cardiac deformation model. Therefore, the medical information processing apparatus 30 according to the first embodiment can predict the blood flow of the subject from the behavior of the heart.

In addition, according to the first embodiment, the acquisition function 352 acquires medical images collected in a plurality of time phases for the heart of the subject and medical information of the subject. Here, the acquisition function 352 acquires a medical image collected for a ventricle of a heart of a subject. In addition, the acquisition function 352 acquires medical information including the sex, blood pressure, height, and weight of the subject. Therefore, the medical information processing apparatus 30 according to the first embodiment can identify the parameter by easily acquirable information.

In addition, according to the first embodiment, the correction function 353 corrects a numerical value based on the subject information acquired by the acquisition function 352. Therefore, the medical information processing apparatus 30 according to the first embodiment can identify the parameter by using more appropriate information.

Furthermore, according to the first embodiment, the correction function 353 corrects the numerical value based on the subject information according to the state of the subject when the subject information is obtained from the subject. Therefore, the medical information processing apparatus 30 according to the first embodiment can identify the parameter by the information in accordance with the state of the patient, and can further improve patient reproducibility.

In addition, according to the first embodiment, the identification function 354 identifies the parameter of each physical model using the subject information for each of the plurality of physical models. Therefore, the medical information processing apparatus 30 according to the first embodiment can optimize the parameter for each patient for each of the plurality of physical models included in the prediction model.

Further, according to the first embodiment, the identification function 354 identifies the parameters of each physical model by inverse analysis using the subject information. Therefore, the medical information processing apparatus 30 according to the first embodiment reduces the calculation cost related to parameter identification and enables clinical application.

### Other Embodiments

In the above-described embodiment, a case where the prediction model 341 includes a drug-ion channel molecular simulation, an electrophysiological simulation of a cell, a contraction simulation of a cardiomyocyte sarcomere, a heartbeat simulation, and a systemic blood flow simulation has been described (FIG. 3). However, the embodiment is not limited thereto, and the prediction model 341 may include a heartbeat simulation and at least one of a drug-ion channel molecular simulation, an electrophysiological simulation of a cell, and a contraction simulation of a cardiomyocyte sarcomere.

Furthermore, in the above-described embodiment, the case of identifying the parameters of all the simulations (physical models) constituting the prediction model 341 based on the subject information has been described. However, the embodiment is not limited thereto, and parameters of only some simulations (physical models) may be identified based on the subject information. Furthermore, an actual measurement value or a predicted value may be applied to some simulations (physical models) constituting the prediction model 341. That is, the prediction model 341 may include a configuration other than the physical model. For example, the prediction model 341 may be configured such that at least one of a molecular dynamic model, an electrophysiological model, a cross-bridge motion model, and a cardiac deformation model is replaced with an actual measurement value, an actual measurement formula, or another predicted value by another prediction model including an artificial intelligence (AI) prediction model. As a result, the medical information processing apparatus 30 can perform prediction in accordance with the state of the patient even in a case where the subject information is partially insufficient.

Furthermore, in the first embodiment described above, a case where each process is performed by an operation via the input interface 32 of the medical information processing apparatus 30 has been described. However, the embodiment is not limited thereto, and the medical information processing apparatus 30 may execute processing in response to an input from the terminal apparatus 20. That is, it is possible to cause the medical information processing apparatus 30 to perform each processing by the operation of the operator via the input interface of the terminal apparatus 20.

Note that, in the above-described embodiment, an example has been described in which the acquisition unit, the correction unit, and the identification unit in the present specification are implemented by the acquisition function, the correction function, and the identification function of the processing circuitry, respectively, but the embodiment is not limited thereto. For example, in addition to being implemented by the acquisition function, the correction function, and the identification function described in the embodiment, the acquisition unit, the correction unit, and the identification unit in the present specification may be implemented by only hardware, only software, or a mixture of hardware and software.

Furthermore, the term “processor” used in the description of the above-described embodiment means, for example, a central processing unit (CPU), a graphics processing unit (GPU), or a circuit such as an application specific integrated circuit (ASIC) or a programmable logic device (for example, a simple programmable logic device (SPLD), a complex programmable logic device (CPLD), and a field programmable gate array (FPGA)). Here, instead of storing the program in the storage circuit, the program may be directly incorporated in the circuit of the processor. In this case, the processor implements the function by reading and executing the program incorporated in the circuit. In addition, each processor of the present embodiment is not limited to a case where each processor is configured as a single circuit, and a plurality of independent circuits may be combined to be configured as one processor to implement the functions.

Here, a medical information processing program executed by the processor is provided by being incorporated in advance in a read only memory (ROM), a storage circuit, or the like. Note that this program may be provided as a computer program product by being recorded in a non-transitory computer-readable storage medium such as a compact disk (CD)-ROM, a flexible disk (FD), a CD-Recordable (R), or a digital versatile disk (DVD) as a file in an installable format or an executable format in these devices. Furthermore, this program may be stored on a computer connected to a network such as the Internet, and may be provided or distributed as a computer program product by being downloaded via the network. For example, this program is configured by a module including each processing function described above. As actual hardware, the CPU reads the medical image processing program from the storage medium such as the ROM and executes the medical image processing program, whereby each module is loaded on the main storage device and generated on the main storage device.

In addition, in the above-described embodiment and modifications, each component of each apparatus illustrated in the drawings is functionally conceptual, and does not necessarily need to be physically configured as illustrated in the drawings. That is, a specific form of distribution or integration of each apparatus is not limited to the illustrated form, and all or a part thereof can be functionally or physically distributed or integrated in any unit according to various loads, usage conditions, and the like. Furthermore, all or any part of each processing function performed in each apparatus can be implemented by a CPU and a program analyzed and executed by the CPU, or can be implemented as hardware by wired logic.

In addition, among the processes described in the embodiment and the modifications described above, all or a part of the processes described as being performed automatically can be manually performed, or all or a part of the processes described as being performed manually can be automatically performed by a known method. In addition, the processing procedure, the control procedure, the specific name, and the information including various data and parameters illustrated in the document and the drawings can be changed at will unless otherwise specified.

Hereinafter, the results of studies verifying the validity of the above-described embodiments and modifications will be described.

### Abstract

We studied an in silico model that envisioned personalized medicine into medication plans that play an important role in the treatment of heart disease. In recent years, in silico models for predicting cardiac behavior have made various advances, and there are also many research examples of techniques for simulating patient conditions that are key for application to personalized medicine. However, an optimization method of model parameters for reproducing individual patients called personalization has two problems. These are the indefiniteness of the parameter that occurs when the measurement value is small and the large calculation cost. In order to improve the personalization method, this study examined introduction of hierarchical parameter optimization for reducing indefiniteness and reduction of calculation cost. The hierarchical parameter optimization was performed by personalization using corresponding measurement values in each scale in a multiscale myocardial model. Furthermore, the calculation cost is reduced by introducing a cylindrical model in which the left ventricle is simplified. As a result of the test calculation, the possibility of improving the problem of parameter indefiniteness was confirmed, and the calculation cost could be reduced to about 9.7 hours by the general-purpose PC. From these, the validity of the proposed technique for the medication plan assuming personalized medicine was shown.

### I. Introduction

In recent years, with the aging of the population in developed countries, it has become more important to improve the treatment of heart diseases represented by arrhythmia and heart failure [1]. Treatments based on statistical analysis for a large number of patients have shown many benefits so far [2] [3]. On the other hand, the departure from the “one-size-fits-all” type medical treatment suggested by the U.S. President in 2015 tends to expand [3]. The refinement of diagnostic data, including the high-resolution imaging of medical images, is expected to reveal differences in pathological conditions between individual patients. In addition to treatment methods based on statistical analysis, it is anticipated to lead to the provision of personalized precision medicine tailored to each patient [4]. In the treatment of heart disease, improvement of heart function by drugs plays an important role. Medication plans are required to consider pharmacological effects and side effects depending on each patient. Therefore, the establishment of the planning method is considered as one of the keys for the realization of personalized medicine in medication [5].

On the other hand, over the past decade, in silico models for predicting cardiac behavior have made significant progress [6]. From the viewpoint of electrochemical-mechanical properties, studies on multiscale and multiphysical cardiac functions by three-dimensional calculation using a cross-bridge motion model of myocardial sarcomere and fluid structure interconnection technology, such as the research by Land et al., have been conducted [7-10]. In terms of pharmacological effects, Yang et al., Llopis-Lorente et al. reproduced the electrochemical properties of cardiomyocytes for drugs with different arrhythmogenic risks, taking into account gender differences [11-13]. These developments have increased the possibility of clinical application of the in silico model, and are expected to be utilized for prediction of pharmacological effects contributing to the medication plan according to the patient. The application of the in silico model to personalized medicine requires a process of optimizing parameters of a physical model to reproduce a patient state, which is called personalization. Research on this optimization process is also in progress, and one of representative methods is a method based on forward analysis. In this method, a variation range of a parameter is determined based on a statistic to create a database, and a parameter set matching a characteristic of pathological ecology of a patient is specified from the database. Another approach is to utilize inverse analysis. Oida et al. propose a method for optimizing model parameters such that simulation results reproduce characteristics of a patient in personalization of a mitral valve model [15]. However, the existing personalization methods have two challenges. A first challenge is indefiniteness that occurs in parameter optimization when the measurement value is small. The progress of the study described above has made it possible to perform an integrated analysis of multi-scale and multi-physics connecting the elastic motion of the left ventricle from the molecular behavior in the ion channel of cardiomyocytes. However, in a case where the measurement value of the patient state is only a specific spatial scale, there is a possibility that a model parameter in a spatial scale different from the specific spatial scale cannot be uniquely determined.

A second challenge is the calculation cost of personalization. In the forward analysis method, a large number of calculations are required to generate a database, and in the inverse analysis method, a large number of calculations are required to adjust parameters. Recently Jung et al. proposed a multi-fidelity approach to this challenge and showed its validity [16]. Since the calculation cost is one of important factors for clinical application of the in silico model, further progress is expected in the future.

In view of the above discussion, this study proposes improvements in how to personalize a numerical model to plan medication according to a patient. For a numerical model for predicting a pharmacological effect, introduction of hierarchical parameter optimization for reducing indefiniteness and reduction of calculation cost were examined.

### II. Method

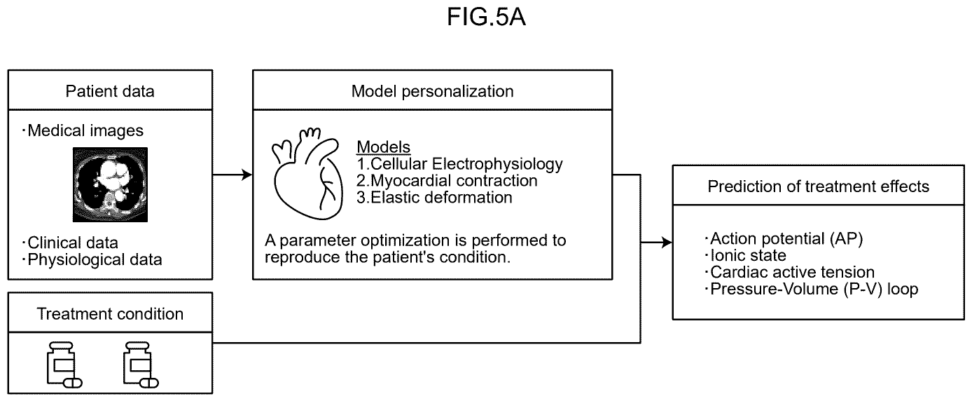

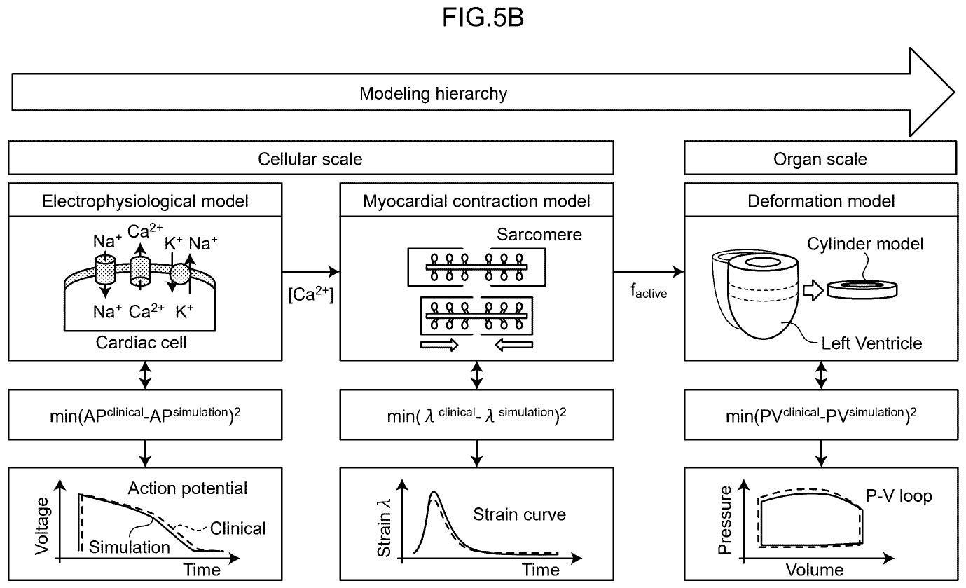

FIGS. 5A and 5B illustrate a calculation model and a workflow for predicting a pharmacological effect on the heartbeat using an approach personalized for each hierarchy constructed in this study. As in the workflow illustrated in FIG. 5A, we first generated a calculation model that reproduces the patient state before medication, and devised a method of predicting the pharmacological effect by reflecting the influence of the drug on some parameters of the model. The calculation model of the cardiac dynamics of this study was composed of three element models. The most microscale element model calculates ionic dynamics and electrical properties in cardiomyocytes. This model includes a physical model of the ion dynamics in the ion channel in which the drug acts and calculates the change in calcium ion dynamics caused by its effect. Using the calcium ion concentration as an input, the myocardial contraction force is calculated based on the myosin cross-bridge motion. In the most macroscale, cardiomyocytes are treated as a continuum and the deformation of the left ventricle is calculated by an elastic deformation model.

The hierarchical personalization approach illustrated in FIG. 5B is characterized in that the above-described three calculation models are personalized by patient measurement values for each model. As the patient measurement value to be applied to the electrochemical model of the cell, a feature amount of an action potential (AP), for example, an action potential duration (APD) is used, and model parameters of the electrochemical model of the cell are optimized such that the calculated value reproduces them. Then, the contraction force model of the ventricular muscle using the calcium ion concentration [Ca2+] as an input constructs a model that reproduces the distortion λ of the ventricular wall that can be measured by the spectral tracking method with respect to the echo image. Finally, for the deformation model of the ventricle, the measurement value of the pressure-volume loop (P-V loop) of the left ventricle is used by using the contraction force f_{active} of the ventricular cells as an input, and model parameters of the deformation are adjusted to reproduce the measurement value. With this approach, the model parameters can be optimized in consideration of the patient state for each hierarchy, rather than selecting up to the microscale model parameters only by the patient's macro P-V loop. Details of the calculation model and parameter optimization of each hierarchy will be described below.

### A. Cell Electrophysiological Model

The ORd model was adopted as a model for simulating the electrochemical characteristics of ventricular myocytes. The ORd model expresses ionic dynamics and electrical characteristics of cells by an ordinary differential equation, and can reproduce action potentials of human cardiomyocytes with high accuracy [18]. As demonstrated in studies of the molecular dynamics of ligand binding to hERG ion channels by Negami et al. [20], the administered drug binds to the pore region in the ion channel and inhibits the flow of ions across the cell membrane. The ORd model considers the flow of 16 ions in the cell membrane, and can adjust each conductance and flux by the input parameters. The conductance is formulated as a function of the indicator IC50 that characterizes the pharmacological effect. This study performed calculations using a program that implemented the ORd model in C++ language.

### B. Myocardial Contraction Model

In this study, the model developed by Land et al. was adopted as a model of the tension of the ventricular muscles [7]. This model has been developed based on a data set of human cardiomyocytes, and can well reproduce the length dependence and strain rate dependence of cardiomyocytes affecting the deformation of the ventricular wall. The mechanical properties of this model are characterized by deformations due to contraction-relaxation movements in the sarcomere, called active parts, and based on the viscoelastic properties of cellular tissues classified as passive parts. For the movement of the sarcomere, a three-state model in which troponin C dynamics, tropomyosin dynamics, and a cross-bridge state are taken into consideration is adopted, and the contraction behavior triggered by the calcium ion concentration is reproduced. Therefore, the contracted state of the sarcomere is affected not only by the deformed state of the active part but also by the distortion of the whole cardiomyocytes including the passive part and the calcium ion concentration related to troponin dynamics. We studied a method of adopting, as an input of the Land model, the calcium ion concentration calculated by the ORd model and the actual measurement value of the temporal change of the distortion by spectral tracking. The Land model was calculated using these input values, and the maximum value of the obtained contraction force was used as an input of the deformation model of the left ventricle. The time term of the ordinary differential equation representing the state change was discretized explicitly using the forward Euler method, and the calculation was performed by the in-house program implemented in the Python language.

### C. Deformation Model of Left Ventricle

The study on deformation models of the heart wall has progressed significantly, and many detailed calculation models have been proposed [9] [21]. Among these, there is a study that uses a relatively large calculation resource compared to a general-purpose PC in order to efficiently perform calculation. Considering that parameter optimization for personalization requires a large number of iterative calculations, we considered introducing a calculation model that can reduce the total calculation time.

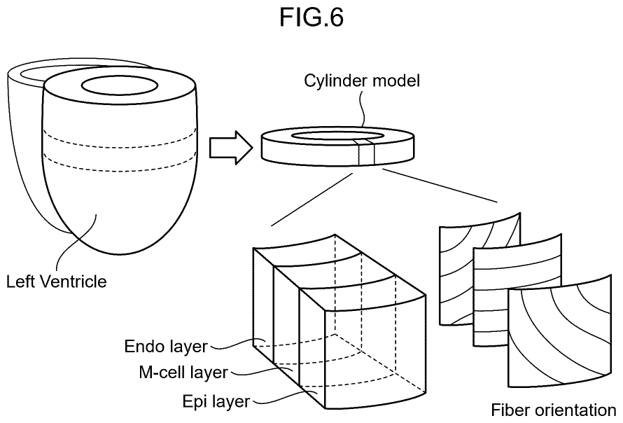

FIG. 6 illustrates a cylindrical model of the left ventricular wall used in this study. The inside of the cylindrical wall had a structure having three layers of electrochemical and mechanical characteristics assuming an endocardium, an M-layer, and an epicardium [22]. Assuming an equator which is a cross section orthogonal to the left ventricular axis and having the largest radius, the diameter of the inside of the cylinder at the initial stage of expansion was set to 6 cm, and the thickness in the radial direction was set to 1 cm. The thicknesses of the three layers in the radial direction were assumed to be the same, and the electrochemical characteristics in the layers were assumed to be uniform. The thickness in the apical direction is positioned as a closure of a cylindrical model, and an approach adopting a ventricular length has been proposed [23]. In this study, it was assumed that the influence was small by reducing the thickness of the cylinder in the apical direction, and the modeling of the potential propagation in the stimulation conduction system was omitted. For the adopted thickness of 1 cm, 10 ms is required at a propagation speed of a general stimulus in the myocardium of 1 m/s [24]. The direction of the myocardial fibers in each layer was assumed to be −60°, 0°, and 60° as inclinations from a plane orthogonal to the left ventricular axis. Since the fibers of the ventricular wall have a continuous structure from the apex toward the upper part of the left ventricle, it is presumed that the deformation by the cylindrical model does not exactly coincide with the actual deformation. On the other hand, a calculation model that simplifies the left ventricular geometry has been adopted in several studies and illustrates its low calculation cost and simplicity of implementation [26]. This study assumed reproduction of qualitative deformation behavior, and adopted this model.

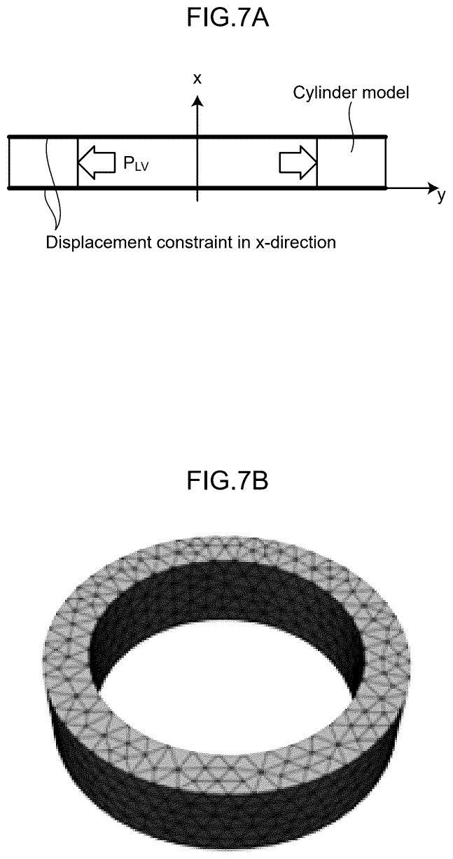

We adopted a toy model in which the dynamics of the myocardial wall is simplified in order to further reduce the calculation cost. FIG. 7A illustrates load conditions and displacement constraint conditions for deformation calculation. A load condition P_{LV} simulating blood flow pressure was applied to the inside of the cylinder to restrain displacement in the x direction on upper and lower surfaces in the x direction. Then, the active force Fa by the sarcomere motion is introduced into the basic equation of the deformation in which the linear elastic body is adopted as the constitution rule (Equation (1)).

$$ρ \frac{{\partial }^{2} u}{\partial {t}^{2}} - Div ( FS ) = {F}_{a} \qquad ( 1 )$$

Here, ρ represents density, u represents a displacement vector, t represents time, F represents a deformation gradient tensor, S represents a second Piola-Kirchhoff stress, and Div represents tensor divergence. The active force uses the maximum value F₀ of the contraction force and the direction vector ξ (Equation (2)).

$${F}_{a} = {F}_{0} f ( t ) ξ \qquad ( 2 )$$

Here, f is a function of temporal change, and in this study, a time distribution of normalized pressure was simply provided.

As described above, since the cylindrical model is different from the actual myocardial shape, there is a phenomenon that is difficult to reproduce. In particular, the deformation of ventricular contraction caused by the three-layer structure of myocardial fibers connecting from the upper ventricle to the apex with a twist is likely a phenomenon difficult to address in a cylindrical model, which exhibits differences in structural rigidity, mechanical conditions, and kinematic conditions due to the extraction of only a portion of this structure. Morishita et al. [26] with a cylindrical model determined model parameters in the evolution of absolute values of active stress to reproduce the desired values of ejection fraction and discussed myocardial strain during cardiac cycles. In this study, with reference to their method, the maximum value F₀ was set by treating only the cylindrical plane direction component, considering providing the active force to reproduce the actual left ventricular P-V loop. In the setting, F₀ is first adjusted so as to reproduce the actual P-V loop under the reference conditions described in chapter 3-B, and F₀ is determined by the contraction force obtained by the Land model for other conditions. We obtained the maximum value of the contraction force by the Land model in the three layers, obtained the ratio of the maximum value to the reference condition, and multiplied F₀ of the reference.

In addition, recent studies on detailed myocardial models have proposed the Fung type constitutive rule, a transversely isotropic hyperelastic material, for the passive part to reproduce mechanical properties. The present study focused on the Proof Of Concept of a hierarchical approach of personalization, and considered first to perform qualitative prediction of pharmacological effects, and adopted a basic model by a constitutive rule.

Finite Element Method (FEM) was used as a deformation calculation method. A tetrahedral primary solid element was employed, and the number of elements of the mesh illustrated in FIG. 7B was 2375. The calculation of the FEM was performed by a program implemented in a program language c++.

### D. Personalization Methods

In the personalization for each hierarchy, the model parameters are optimized so as to reduce the difference between the measurement value and the calculated value. In this study, an action potential was considered as a measurement value used for personalization of the ORd model, and a time-to-peak (tp) response of AP, action potential duration at 90% of repolarization (APD90) were extracted as a feature amount. Then, a loss function regarding a difference between the feature amount and the calculated value was defined and used for comparison (Equation (3)).

$${L}_{ORd} = {( {tp}^{m} - {tp}^{s} )}^{2} + ( APD {90}^{m} - APD {90}^{2} ) \qquad ( 3 )$$

Here, the upper subscript m means a measurement value, and s means a calculated value. As described above, the measurement value of the distortion of the left ventricular wall was adopted for personalization of the Land model. For the deformation model of the left ventricle, a P-V loop was considered as a measurement value, and personalization was performed so that a temporal change in volume can be reproduced. Note that the left ventricular volume V was obtained on the assumption that the shape of the ventricle was a semi-ellipsoid and the inner diameter of the cylindrical model corresponded to the maximum radius thereof. The ventricular length at the end diastole was determined, and it was assumed that the ventricular length ratio changes similarly to the change rate of the cylindrical model inner diameter. A loss function was defined similarly to the ORd model, and was defined as the square of the volume difference between the end-diastolic volume EDV and the end-systolic volume ESV (Equation (4)).

$${L}_{Defo} = \frac{1}{2} \{ {( {EDV}^{m} - {EDV}^{s} )}^{2} + {( {ESV}^{m} - {ESV}^{s} )}^{2} \} \qquad ( 4 )$$

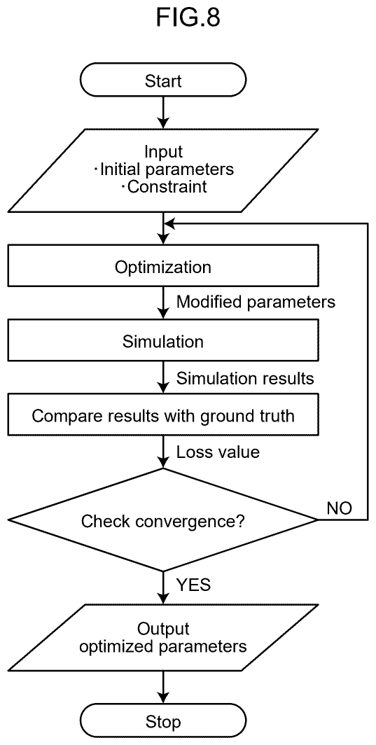

For parameter optimization of the ORd model and the deformation model, an algorithm illustrated in FIG. 8 is adopted. The parameter is adjusted so that the loss function of Equation (3) or Equation (4) is minimized, and iterative calculation is performed until the loss function converges. For the adjustment of the parameters, this study adopted Covariance Matrix Adaptation Evolution Strategy (CMA-ES) [30]. This optimization algorithm has a process of considering the order of the objective function in the selection of the search direction, and there is a possibility that an efficient search can be achieved without calculation of the gradient.

TABLE 1

| Model | Parameter (Symbol) |
|---|---|
| ORd | Max. conductance of fast Na⁺ channels (G_{Na, fast}) |
| model | Max. conductance of late Na⁺ channels (G_{Na, late}) |
| | Max. conductance of inward-rectifier K⁺ channels (G_{K1}) |
| | Max. conductance of rapid delayed rectifier K⁺ channels (G_{Kr}) |
| | Max. conductance of slow delayed rectifier K⁺ channels (G_{Ks}) |
| | Max. conductance of transient outward K⁺ channels (G_{Kto}) |
| | Max. conductance of Na⁺/Ca²⁺ exchangers (G_{NaCa}) |
| | Max. conductance of sarcolemmal Ca²⁺ pumps (G_{pCa}) |
| | Max. conductance of Na⁺/K⁺ ATPase (G_{NaK}) |
| Deforma- | Elasticity |
| tion model | Poisson ratio |

Table 1 (model parameters to be personalized) shows the ORd model parameters and the deformation model parameters personalized. Muszkiewicz et al. state that both ionic current conductance and dynamics can change between individuals, and that the conductance change is mainly affected by the number of ion channels in the cell membrane [31]. Yang et al. have shown a methodology for scaling ion currents by gene expression values that may cause differences in sex [32]. In this study, with reference to those studies on electrochemical models of cells, conductance related to ion channel groups of Na⁺ and K⁺, a Na⁺—Ca²⁺ exchange system, a Ca²⁺ pump, and Na⁺—K⁺ ATPase was adopted as an optimization parameter of the ORd model. The reference value in the optimization was adopted in the literature in which the ORd model was proposed. The left ventricular deformation model optimized the elastic modulus and Poisson's ratio. Reference values were set with reference to the literature, and the elastic modulus was 300 KPa and the Poisson's ratio was 0.4. In both models, the parameter variation range in the optimization was +50% with respect to the reference value.

On the other hand, for personalization of the Land model, a policy of not using the above-described algorithm was adopted. The calculation method of the Land model adopted in this study can be calculated numerically stably under Isometric constraint, but numerical vibration is generated even when the time interval is 1 ns in a case where the constraint is removed and the contraction force is obtained while the strain is temporally developed. The distortion-decay model of the Land model has two scalar temporal evolution equations representing a pre-powerstroke state and a force-generating state, and they are constituted by a term caused by temporal change of distortion and a decay term. Each state increases or decreases with the balance of the two terms, and the time evolution method of distortion is affected by the active force depending on the state. Since the decay term has an exponential behavior, we estimated that when the distortion is calculated from the temporal evolution equation, vibration like alternately advanced and retarded type is likely to occur. Therefore, in this study, the temporal evolution equation of the distortion was not solved, and the time derivative value of the distortion was input to define one behavior, thereby improving the numerical stability. The input time derivative value of the distortion was calculated from the patient-specific measurement value, and the literature value [7] was simply adopted as the model parameter. Even if the temporal change of the distortion is given, the scalar representing the state in all the calculations performed in this study is temporally evolved within a physically matching value range.

### III. Results

### A. Operation Verification of Physical Model for Each Hierarchy

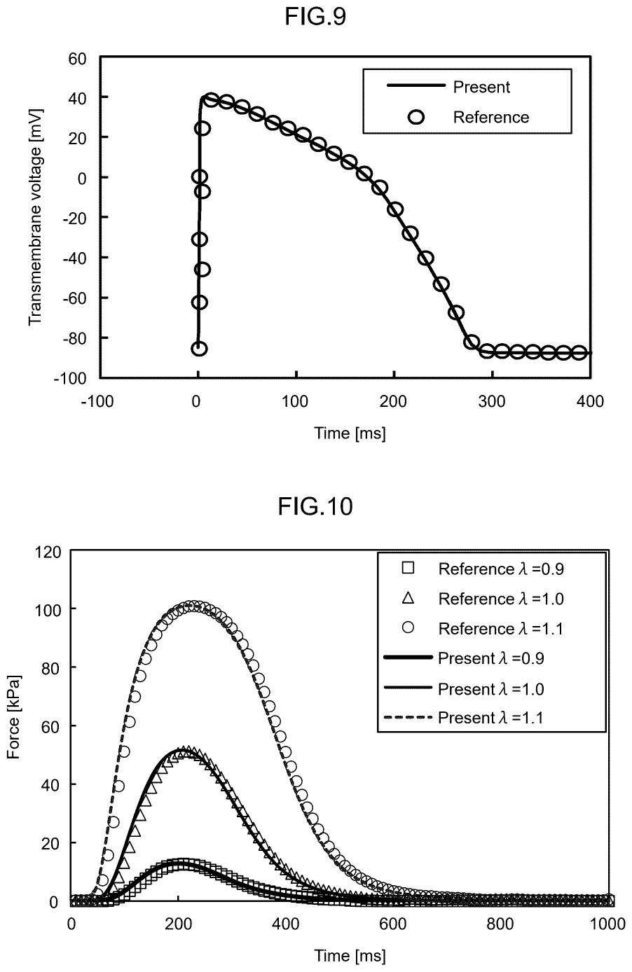

First, verification for confirming the calculation operation of each hierarchical model was performed. The calculation of the ORd model for determining the electrochemical characteristics of cells was compared with the calculation results described in the literature by Ohara et al. FIG. 9 illustrates a comparison between the results of this calculation and action potentials read from the literature. Both are in good agreement, suggesting that the calculation program of the ORd model used in this study is correctly implemented.

Next, the calculation result of the Land model for determining the active force of cardiomyocytes was compared with the literature [7] by Land et al. FIG. 10 illustrates a comparison of contraction force histories under isometric conditions. Without depending on the distortion, both were considered to be in good agreement, suggesting that the in-house code of the Land model is operating correctly.

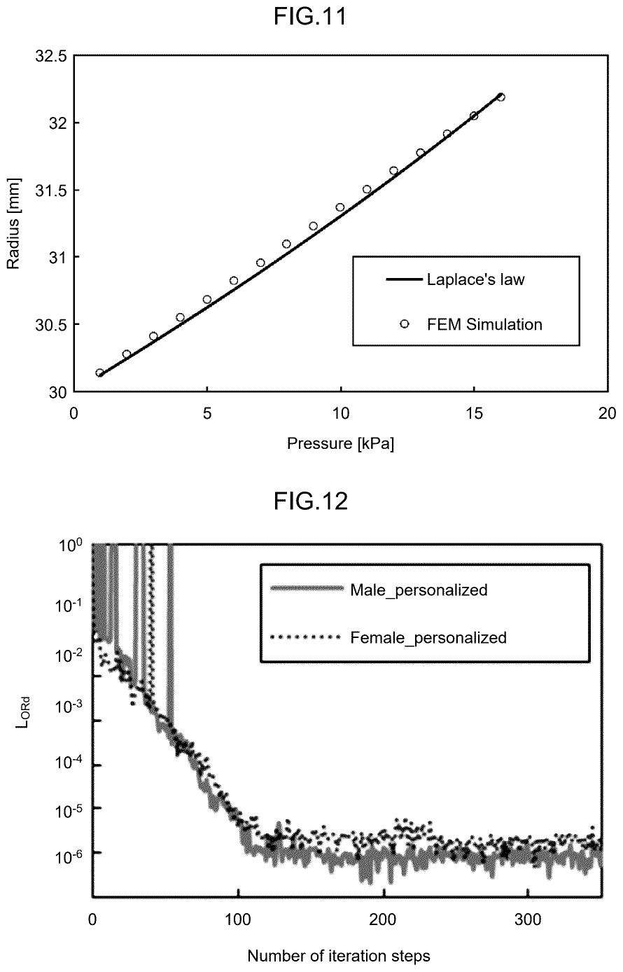

At the end of the operation verification, for the cylindrical model of the left ventricular deformation, the calculation results were compared with Laplace law. Laplace law can analytically determine the cylindrical shape assuming a static balance between internal pressure and elastic deformation. The active force was set to zero according to an assumption, and dynamic analysis was performed until reaching a steady state. FIG. 11 illustrates the present calculation result regarding the pressure and the cylinder inner diameter and the calculation result by Laplace law. The pressure was calculated based on a maximum pressure of 120 mmHg of a general left ventricle of a human. Both are in good agreement, suggesting that the elastic deformation corresponding to the passive part of the deformation model is implemented correctly.

### B. Verification of Hierarchical Personalization Method

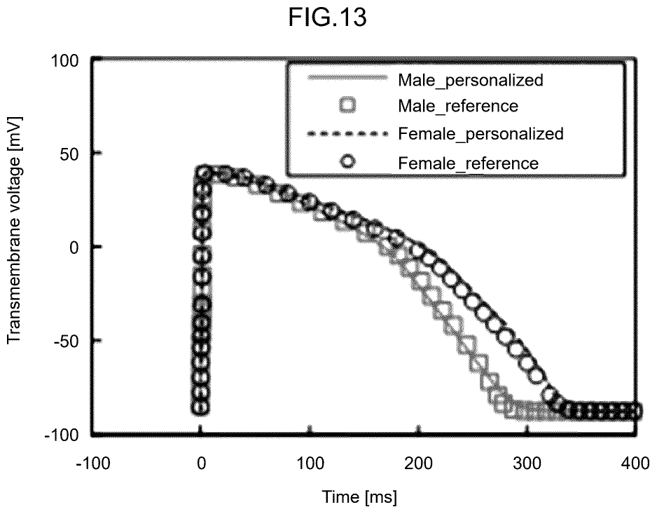

In order to verify the operation of the proposed personalization method, patient measurement values were virtually set, and personalization was attempted in hierarchical order. First, a virtual patient action potential is given, and personalization of the ORd model is performed. In general, trials with male and female action potentials having differences in characteristics were considered, and virtual measurement values were generated using the ORd model with reference to the literature by Yang et al. FIG. 12 illustrates transition of the loss function of the calculation result. From FIG. 12, the loss function reaches a convergence state in about 100 generations, and the error of the feature amount with respect to time decreases to 10⁻⁷ corresponding to 0.3 μs. Note that in this algorithm, the loss function value is set to a huge value when the parameter value falls outside the constraint range, and thus this occurs at the beginning of the generation update. FIG. 13 illustrates a comparison between the personalized action potential and the virtual measurement value. As assumed from the convergence state of the loss function, both are in good agreement.

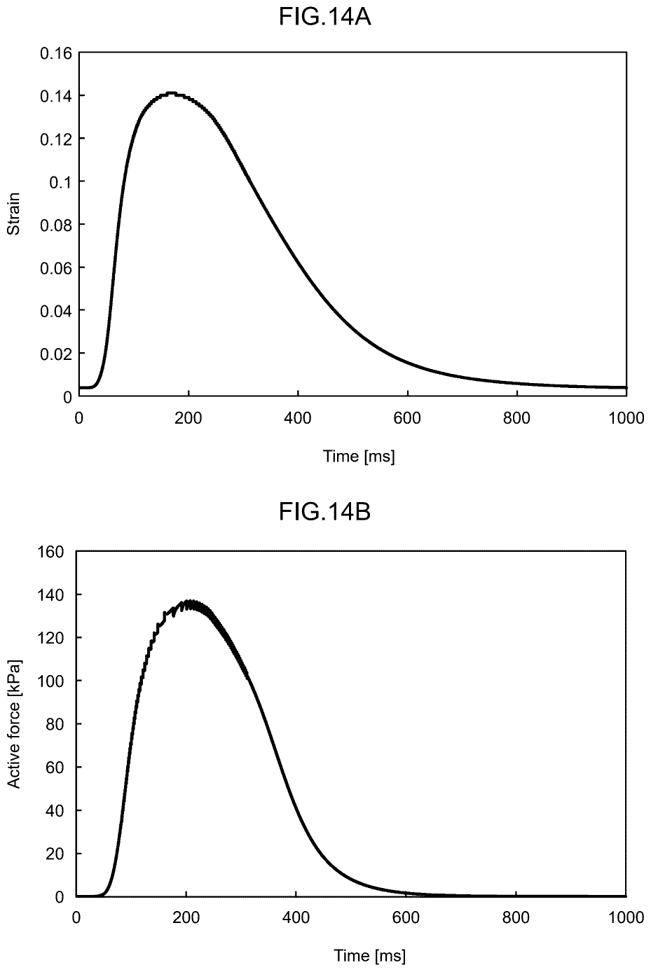

Next, virtual personalization of the Land model was performed. FIG. 14A illustrates a temporal change of the input virtual distortion and FIG. 14B illustrates a time history of the calculated active force. Although the test calculation is a virtual input value, the patient's distortion distribution can be reflected in the physical model by using the present approach. Although slight vibration was observed in the vicinity of 200 ms close to the peak value of the calculated active force, the calculated active force could be calculated without numerically collapsing.

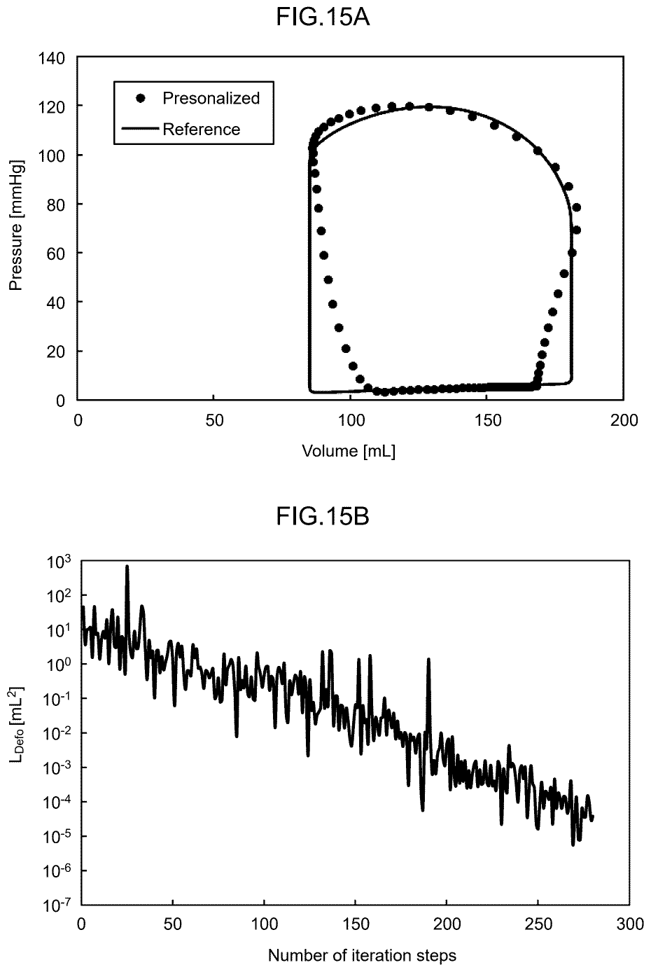

Subsequently, virtual personalization of the deformation model was performed. FIG. 15A illustrates a comparison between the calculated value and the P-V loop of the patient virtually set, and FIG. 15B illustrates a transition of the loss function. In this study, the active force maximum value F₀ is adjusted using this calculation as a reference condition. The virtual patient data and the calculated values showed qualitative agreement, and the patient-specific volume change amount could be reproduced. The loss function showed a constant decreasing tendency, and the radius error of the cylindrical model at the finally reached loss value was 1.4 mm.

### C. Consideration on Validity of Hierarchical Personalization

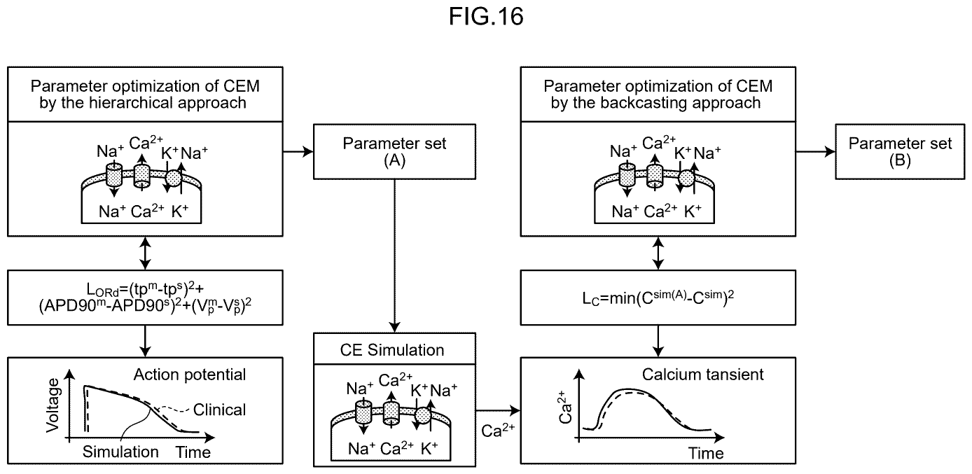

Considering the current clinical environment, measurement of macroscopic cardiac features represented by CT images may be included in a diagnostic workflow, while measuring action potentials of cardiomyocytes is not known to the authors. The electrocardiogram is a valid means for evaluating the electrical dynamics of the living body, but cannot measure the electrochemical dynamics of each cell. From that viewpoint, acquiring the measurement corresponding to the physical phenomenon handled by the hierarchical model of the microscale tends to be insufficient compared to that of the macroscale. If there is no measurement value corresponding to the calculation model to be personalized, but there is the calculation model of the higher scale, an approach of backcasting from the personalization result of the higher model is conceivable. For example, the method is a method of personalizing a Land model that is a higher order of the ORd model by using macro measurements, and personalizing by setting the calcium ion distribution used therein to an objective function so that the ORd model satisfies. In this study, in order to discuss validity of proposed hierarchical personalization, verification using the ORd model illustrated in FIG. 16 was performed. In the verification, first, parameter optimization is performed with the action potential as an objective function, and as a result, calcium ion variation is obtained. Then, parameter optimization is performed by changing the variation to an objective function, and the action potential is calculated by the obtained parameter. The difference between these action potentials was examined and considered.

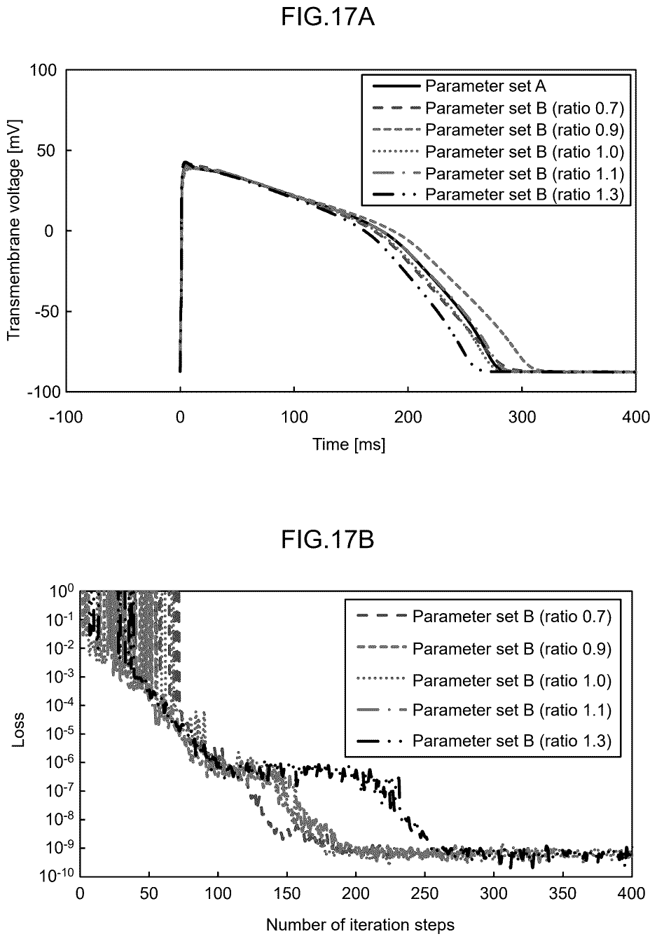

FIGS. 17A and 17B illustrate a comparison of action potentials of verification results and transition of a loss function. We were concerned about the parameter initial value dependency to be optimized, and the optimization was performed for 5 conditions in which the average initial value was set to 0.7, 0.9, 1.0, 1.1, and 1.3 times the reference value. As is clear from the action potential illustrated in FIG. 17A, the result of the parameter B optimized by assuming the backcast approach can confirm the initial value dependency on the repolarized phase. When the time at which the stationary phase is reached is compared with the calculation result of the parameter A, it is +30 ms and −20 ms under the conditions of the magnifications of the initial values of 0.9 and 1.3, respectively. This variation range is similar to 40 ms, which is an example of the difference caused by the action potentials of the male and the female illustrated in FIG. 13. In addition, the action potential according to parameter A transits to the stationary phase at about 280 ms, and the above-described initial value dependency variation range of 50 ms corresponds to 18% thereof. On the other hand, the transition of the loss function in the optimization in which the parameter B is obtained illustrated in FIG. 17B confirms a plateau in the middle of the calculation of the magnification 1.3 of the initial average value, but reaches a sufficiently small convergence value in the 400 generation. Therefore, it can be seen that the initial value dependency caused in the APD by the parameter B is not an error caused by the optimization failure.

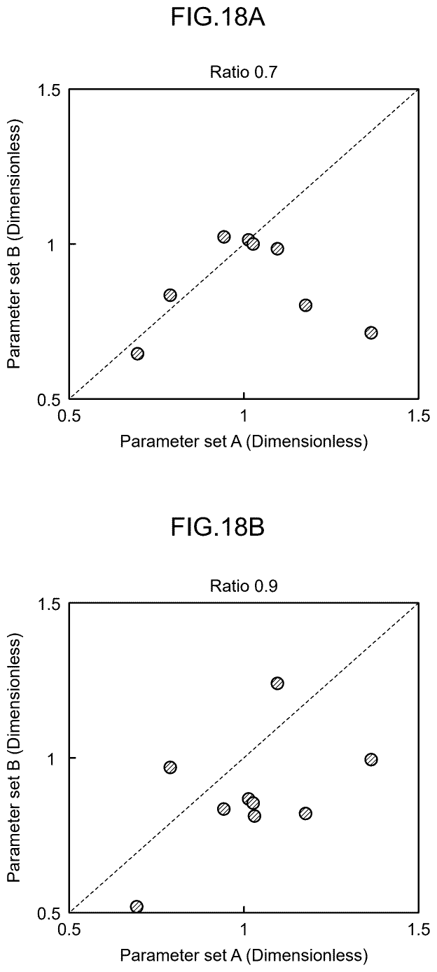

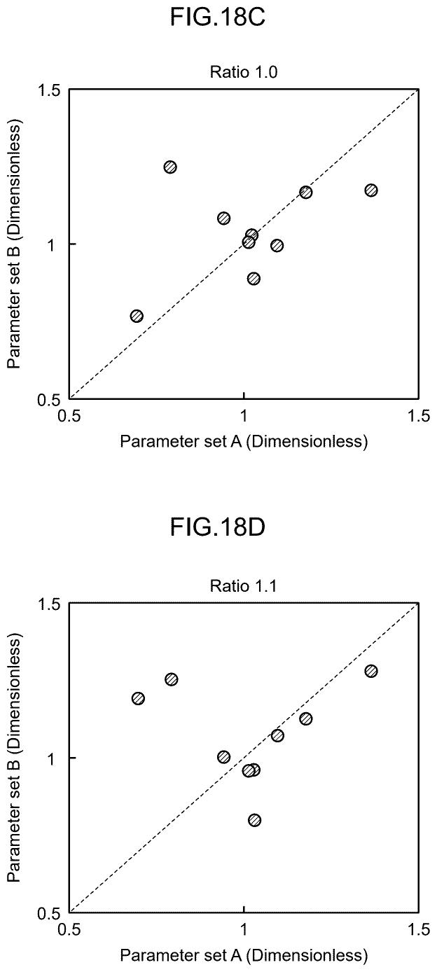

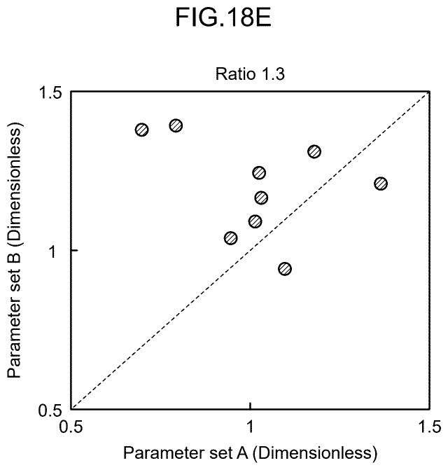

Next, a correlation between two parameter sets personalized by different approaches was examined. FIGS. 18A to 18E illustrate results obtained by plotting correlations of normalized parameter values for each initial value condition. Both parameter sets are optimized within a range of values with respect to a set reference value and have a distribution. In the magnification 0.9 in which there was a difference in the time reaching the stationary phase, the parameter set B has a parameter in which the lower limit value of the value range is taken, but it was not possible to confirm a specific correlation between the 0.9 and 1.3 conditions as a whole. Under the conditions performed this time, in the backcast approach, a method for selecting the initial magnification condition that matches the action potential according to the parameter A from only the parameter distribution has not been found. The backcast approach has an advantage of easily obtaining a measurement value, but this parameter indefiniteness may be a problem. Therefore, from the viewpoint of reproducibility of the individual patient state, the proposed technique of personalizing each hierarchy by using the measurement value is considered to be validity.

### D. Application-Prediction of Pharmacological Effects of Dofetilide

In order to consider the prediction performance of the pharmacological effect of the proposed technique, a medication simulation was performed on the virtual male- and female-patient models personalized in Section III-B. For the virtual medication, Dofetilide classified as high risk in the TdP category of The Comprehensive in Vitro Proarrhythmia Assay (CiPA) was set [35]. Similar to the study on pharmacological effects using the in silico model [13], the influence of drugs is reflected in the calculation model as ion channel inhibition. This study reflected the input model parameters IC50 and Hill coefficients for 7 ion currents, i.e., I_{Na}, Iₜₒ, I_{CaL}, I_{NaL}, I_{Kr}, I_{Ks}, and I_{K1}=I, as 2 times the maximum blood concentration Cₘₐₓ. The influence of the drug on the parameters of the remaining ORd model, the Land model parameters, and the parameters of the deformation model was assumed to be negligible, and personalized parameters were used.

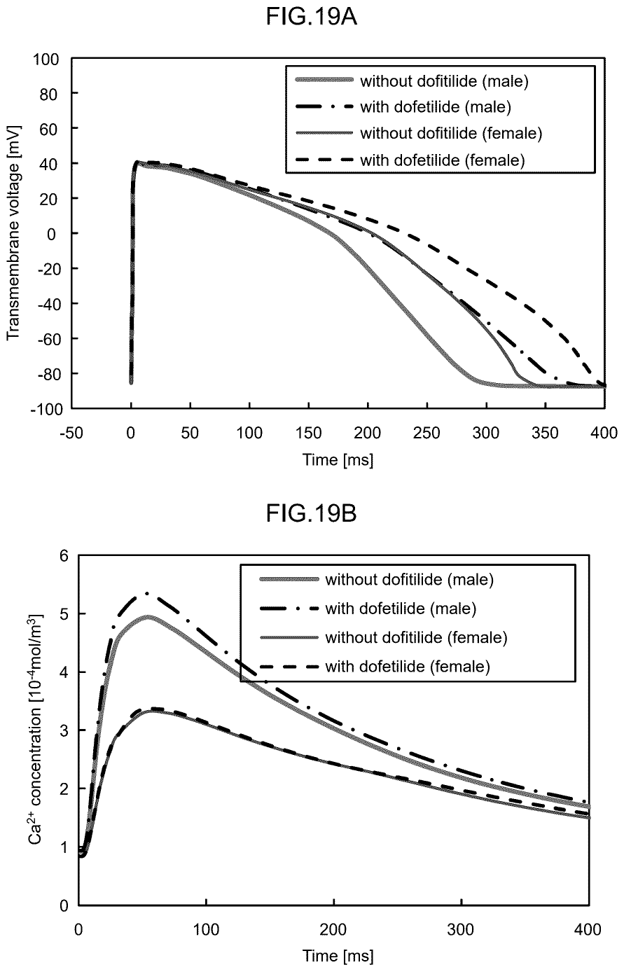

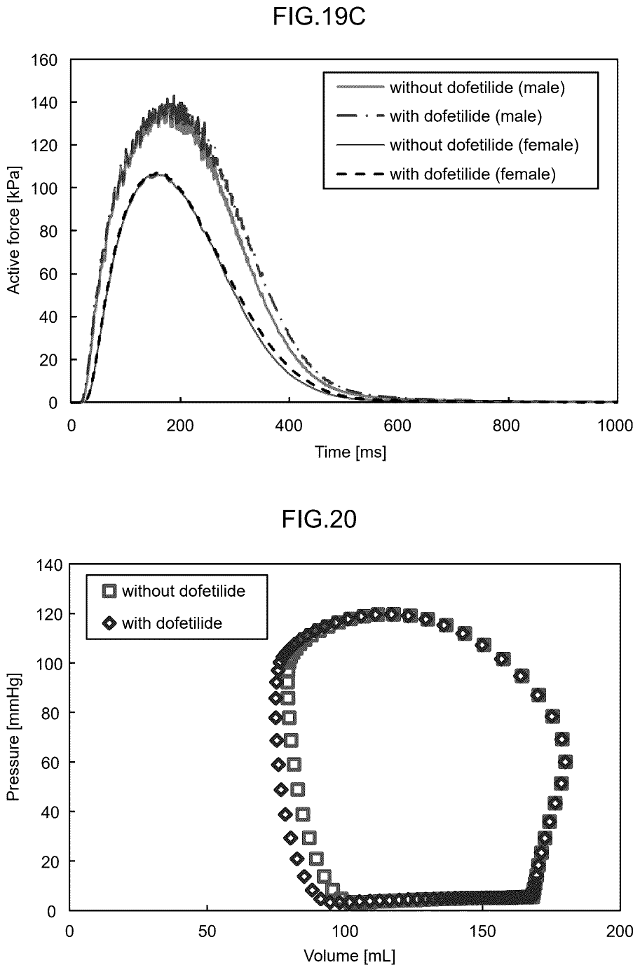

FIGS. 19A to 19C illustrate comparison of electrochemical kinetics and active force of cardiomyocytes in the Endo layer before and after medication. Dofetilide, known as a hERG blocker, inhibits the potassium ion current in the third phase and causes QT prolongation [12]. In the action potential illustrated in FIG. 19A, extension of the third phase is observed due to the pharmacological effect in both virtual male patients and female patients, and such an effect of Dofetilide is qualitatively reproduced. Yang et al. conducted pseudo ECG calculation and reported that the QT-prolongation at the Cₘₐₓ was about 80 ms, but the prolongation time estimated using the time to reach the stationary phase for this calculation was 75 ms and 70 ms for the male- and female-conditions, respectively, and was qualitatively reproduced. The calcium concentration illustrated in FIG. 19B tends to be relatively large due to the pharmacological effect, and the effect is particularly remarkable under male conditions. The calcium concentration peak in the male condition in which a difference occurs depending on the presence or absence of medication occurs in the vicinity of 55 ms, and it is presumed that the inhibitory effect of I_{Na}, Iₜₒ, I_{CaL}, and I_{NaL} acts in the vicinity of this phase. Tomek et al. report that the maximum calcium concentration increases when sodium current is inhibited by 50% in the ORd model, and the trend is consistent with the present calculation result [36]. Note, however, that Tomek et al. point to the reproducibility of the ORd model for the negative inotropy observed in sodium blockers, and that the peak value increase in pharmacological conditions may be too high compared with actual values. On the other hand, the calcium concentration in the female condition has a relatively smaller pharmacological effect than that in the male condition. This small influence means that the pharmacological effect is less likely to appear on the myocardial deformation when the electrochemical characteristics in the cylindrical model are assumed to be uniform. On the other hand, such deformation behavior is qualitatively inconsistent with the medical findings that in Dofetilide, women show lower clearance than men. This cause indicates not only the possibility of the influence of the prediction performance of the ORd model described above but also the limitation of the assumption introduced in this model. Yang et al. performed two-dimensional potential propagation calculation of 5 cm square, and showed that a difference occurs between the Endo phase and the Epi phase in the propagation state at the tissue scale. This phase difference in the propagation state leads to separation of the myocardial contraction from the control state, and is considered to be a cause of the onset of arrhythmia. From this, it is estimated that the assumption that the electrochemical characteristics in the cylindrical model are uniform is too strong depending on patient conditions and drugs. As a future work, we plan to study a method of calculating tissue-level stimulation conduction while reducing calculation cost. Next, FIG. 19C illustrates a time history of the myocardial contraction force. As previously discussed, myocardial contraction is triggered by calcium concentration in the framework of the present study. Therefore, the influence of the drug was more remarkable in men as with the calcium concentration, and the contraction force generally increased in the male condition, and slightly increased in the third phase of the action potential in the female condition.

Finally, FIG. 20 illustrates a calculation result of the deformation model using the active force under a male condition in which a pharmacological effect is further generated. In the P-V loop obtained by the present modification calculation assuming virtual medication, the systolic volume tended to decrease as compared with that before the medication due to the increased active force. As described above, one cause of the arrhythmia is the deviation of the endocardium and the epicardium from the control state, and this model cannot consider the deviation. If a detailed full model including a three-dimensional stimulation conduction system is used, the ventricular behavior including the endocardium and the epicardium can be predicted. However, the present model was able to show that at least the active force changed by the pharmacological effect may affect the left ventricular deformation under the assumed conditions.

### E. Application-Calculation Time

Table 2 (calculation time in calculation of pharmacological effect) shows calculation time of each hierarchy in personalization and medication simulation. In the calculation time when the general-purpose PC having the number of clocks of 3.2 GHZ CPU was used, the personalization time ratio was 99.5% on average, and the total calculation time was 9.7 hours on average. Note that the personalization of the parameter-optimized ORd model and the deformation model was calculated by openMP parallel of one core and four cores according to the recommended sampling number of the CMA-ES. All other calculation times listed in Table 2 are calculation by single core.

TABLE 2

| | Personalization | | | Prediction of treatment effects | | | |
|---|---|---|---|---|---|---|---|
| | ORd | Land | Cylinder | ORd | Land | Cylinder | Total |
| | model | model | model | model | model | model | time |
| Case | (sec) | (sec) | (sec) | (sec) | (sec) | (sec) | (sec) |
| Male | 26614 | 2 | 8555 | 157 | 2 | 34 | 35364 |
| Female | 25821 | 2 | 8646 | 129 | 2 | 33 | 34633 |

With recent remarkable progress, detailed electrochemical-, solid deformation-, or physical models combining them have been constructed. According to the paper of Caballero et al. in 2018, it is reported that the deformation of the mitral valve of 2 beats using the three-dimensional FSI and the blood flow calculation took 240 hours using 64 cores [38]. Bucelli et al. reported in 2023 that in simulation of the left heart in consideration of electrophysiology, mechanics, and fluid dynamics, it took about 19 hours for 192 parallel calculations of 1 beat [39]. In order to optimize these calculation model parameters by iterative calculation, it is necessary to further add a calculation cost. Jung et al. proposed Multi-Fidelity Approach, and achieved calculation cost reduction of parameter optimization of a three-dimensional calculation model [16]. In addition, Isotani et al. propose a method of reducing the calculation cost by treating only the ventricle in three dimensions [40]. The proposed technique showed the possibility of predicting the therapeutic effect in 9.7 hours while using up to 4 cores. In the future, not only the physical model but also the technology for reducing the calculation cost are expected to be developed.

### IV. Conclusions

A method for generating a personalized model in an in silico model for predicting pharmacological effects contributing to a medication plan was constructed. A hierarchical personalization approach was devised to mitigate parameter indefiniteness that may occur when patient measurements are low. In addition, a calculation model for reducing the processing time of personalization is adopted. The verification calculation suggests the usefulness of the proposed technique as compared with the personalization method of backcasting from the upper level of the hierarchy. Personalization and pharmacological effect prediction for virtual patient data were performed to qualitatively reproduce previous findings regarding QT prolongation. In addition, it was shown that a pharmacological effect occurs in the myocardial contraction force and the left ventricular behavior simulated by a cylinder. The total calculation time required for personalization and pharmacological effect prediction was about 9.7 hours when 4 cores of a general-purpose PC were used.

### REFERENCES

[1] North B J, Sinclair D A. The intersection between aging and cardiovascular disease. Circ Res. 2012 Apr. 13; 110(8):1097-108. doi: 10.1161/CIRCRESAHA. 111.246876.
[2] Strait J B, Lakatta E G. Aging-associated cardiovascular changes and their relationship to heart failure. Heart Fail Clin. 2012 January; 8(1):143-64. doi: 10.1016/j.hfc.2011.08.011.
[3] Ponikowski P, Voors A A, Anker S D, Bueno H, Cleland J G F, et al.; ESC Scientific Document Group. 2016 ESC Guidelines for the diagnosis and treatment of acute and chronic heart failure: The Task Force for the diagnosis and treatment of acute and chronic heart failure of the European Society of Cardiology (ESC) Developed with the special contribution of the Heart Failure Association (HFA) of the ESC. Eur Heart J. 2016 Jul. 14; 37(27):2129-2200. doi: 10.1093/eurheartj/ehw128. Epub 2016 May 20. Erratum in: Eur Heart J. 2018 Mar. 7; 39(10):860. doi: 10.1093/eurheartj/ehw383.
[4] Juengst E, McGowan M L, Fishman J R, Settersten R A Jr. From “Personalized” to “Precision” Medicine: The Ethical and Social Implications of Rhetorical Reform in Genomic Medicine. Hastings Cent Rep. 2016 September; 46(5):21-33. doi: 10.1002/hast.614.
[5] Han X, Zhou Y, Liu W. Precision cardio-oncology: understanding the cardiotoxicity of cancer therapy. NPJ Precis Oncol. 2017 Sep. 12; 1(1):31. doi: 10.1038/s41698-017-0034-x.
[6] Niederer S A, Lumens J, Trayanova N A. Computational models in cardiology. Nat Rev Cardiol. 2019 February; 16(2):100-111. doi: 10.1038/s41569-018-0104-y.
[7] Land S, Park-Holohan S J, Smith N P, Dos Remedios C G, Kentish J C, Niederer S A. A model of cardiac contraction based on novel measurements of tension development in human cardiomyocytes. J Mol Cell Cardiol. 2017 May; 106:68-83. doi: 10.1016/j.yjmcc.2017.03.008.
[8] Relan J, Chinchapatnam P, Sermesant M, Rhode K, Ginks M, Delingette H, Rinaldi C A, Razavi R, Ayache N. Coupled personalization of cardiac electrophysiology models for prediction of ischaemic ventricular tachycardia. Interface Focus. 2011 Jun. 6; 1(3):396-407. doi: 10.1098/rsfs.2010.0041.
[9] Santiago A, Aguado-Sierra J, Zavala-Aké M, Doste-Beltran R, Gómez S, Arís R, Cajas J C, Casoni E, Vázquez M. Fully coupled fluid-electro-mechanical model of the human heart for supercomputers. Int J Numer Method Biomed Eng. 2018 December; 34(12): e3140. doi: 10.1002/cnm.3140.
[10] Okada J, Yoshinaga T, Kurokawa J, Washio T, Furukawa T, Sawada K, Sugiura S, Hisada T. Screening system for drug-induced arrhythmogenic risk combining a patch clamp and heart simulator. Sci Adv. 2015 May 1; 1(4): e1400142. doi: 10.1126/sciadv.1400142.
[11] Yang P C, Perissinotti L L, López-Redondo F, Wang Y, DeMarco K R, Jeng M T, Vorobyov I, Harvey R D, Kurokawa J, Noskov S Y, Clancy C E. A multiscale computational modelling approach predicts mechanisms of female sex risk in the setting of arousal-induced arrhythmias. J Physiol. 2017 Jul. 15; 595(14):4695-4723. doi: 10.1113/JP273142.
[12] Yang P C, DeMarco K R, Aghasafari P, Jeng M T, Dawson J R D, Bekker S, Noskov S Y, Yarov-Yarovoy V, Vorobyov I, Clancy C E. A Computational Pipeline to Predict Cardiotoxicity: From the Atom to the Rhythm. Circ Res. 2020 Apr. 10; 126(8):947-964. doi: 10.1161/CIRCRESAHA.119.316404.
[13] Llopis-Lorente J, Baroudi S, Koloskoff K, Mora M T, Basset M, Romero L, Benito S, Dayan F, Saiz J, Trenor B. Combining pharmacokinetic and electrophysiological models for early prediction of drug-induced arrhythmogenicity. Comput Methods Programs Biomed. 2023 December; 242:107860. doi: 10.1016/j.cmpb.2023.
[14] Okada J I, Yoshinaga T, Kurokawa J, Washio T, Furukawa T, Sawada K, Sugiura S, Hisada T. Arrhythmic hazard map for a 3D whole-ventricle model under multiple ion channel block. Br J Pharmacol. 2018 September; 175(17):3435-3452. doi: 10.1111/bph.14357.
[15] Ooida J, Kiyohara N, Noguchi H, Oguchi Y, Nagane K, Sakaguchi T, Aoyama G, Shige F, Chapman J V, Asami M, Kofoed K F, Pham M H C, Suzuki K. An In Silico Model for Predicting the Efficacy of Edge-to-Edge Repair for Mitral Regurgitation. J Biomech Eng. 2024 Feb. 1; 146(2):021004. doi: 10.1115/1.4064055.
[16] Jung A, Gsell M A F, Augustin C M, Plank G. An Integrated Workflow for Building Digital Twins of Cardiac Electromechanics-A Multi-Fidelity Approach for Personalising Active Mechanics. Mathematics (Basel). 2022 Mar. 4; 10(5):823. doi: 10.3390/math10050823.
[17] O'Hara T, Virág L, Varró A, Rudy Y. Simulation of the undiseased human cardiac ventricular action potential: model formulation and experimental validation. PLOS Comput Biol. 2011 May; 7(5): e1002061. doi: 10.1371/journal.pcbi.1002061.
[18] Passini E, Britton O J, Lu H R, Rohrbacher J, Hermans A N, Gallacher D J, Greig R J H, Bueno-Orovio A, Rodriguez B. Human In Silico Drug Trials Demonstrate Higher Accuracy than Animal Models in Predicting Clinical Pro-Arrhythmic Cardiotoxicity. Front Physiol. 2017 Sep. 12; 8:668. doi: 10.3389/fphys.2017.00668.
[19] Negami T, Terada T. Calculations of the binding free energies of the Comprehensive in vitro Proarrhythmia Assay (CiPA) reference drugs to cardiac ion channels. Biophys Physicobiol. 2023 Mar. 25; 20(2): e200016. doi: 10.2142/biophysico.bppb-v20.0016.
[20] Negami T, Araki M, Okuno Y, Terada T. Calculation of absolute binding free energies between the hERG channel and structurally diverse drugs. Sci Rep. 2019 Nov. 12; 9(1):16586. doi: 10.1038/s41598-019-53120-6.
[21] Gerach T, Schuler S, Fröhlich J, Lindner L, Kovacheva E, Moss R, Wulfers E M, Seemann G, Wieners C, Loewe A. Electro-Mechanical Whole-Heart Digital Twins: A Fully Coupled Multi-Physics Approach. Mathematics. 2021; 9(11):1247. https://doi.org/10.3390/math9111247
[22] Ramírez W A, Gizzi A, Sack K L, Guccione J M, Hurtado D E. In-silico study of the cardiac arrhythmogenic potential of biomaterial injection therapy. Sci Rep. 2020 Jul. 31; 10(1):12990. doi: 10.1038/s41598-020-69900-4.
[23] Genet M, Diaz J, Chapelle D, Moireau P. Reduced left ventricular dynamics modeling based on a cylindrical assumption. Int J Numer Method Biomed Eng. 2023 November; 39(11): e3711. doi: 10.1002/cnm.3711.
[24] Laske, T. G., Shrivastav, M., Iaizzo, P. A. (2015). The Cardiac Conduction System. In: Iaizzo, P. (eds) Handbook of Cardiac Anatomy, Physiology, and Devices. Springer, Cham. https://doi.org/10.1007/978-3-319-19464-6_13
[25] Pravdin S F, Berdyshev V I, Panfilov A V, Katsnelson L B, Solovyova O, Markhasin V S. Mathematical model of the anatomy and fibre orientation field of the left ventricle of the heart. Biomed Eng Online. 2013 Jun. 18; 12:54. doi: 10.1186/1475-925X-12-54.
[26] Morishita T, Takeishi N, Ii S, Wada S. Effects of Left Ventricular Hypertrophy and Myocardial Stiffness on Myocardial Strain Under Preserved Ejection Fraction. Ann Biomed Eng. 2021 July; 49 (7): 1670-1687. doi: 10.1007/s10439-020-02706-7.
[27] Owen B, Bojdo N, Jivkov A, Keavney B, Revell A. Structural modelling of the cardiovascular system. Biomech Model Mechanobiol. 2018 October; 17 (5): 1217-1242. doi: 10.1007/s10237-018-1024-9.
[28] Genet M, Lee L C, Baillargeon B, Guccione J M, Kuhl E. Modeling Pathologies of Diastolic and Systolic Heart Failure. Ann Biomed Eng. 2016 January; 44 (1): 112-27. doi: 10.1007/s10439-015-1351-2.
[29] Margara F, Wang Z J, Levrero-Florencio F, Santiago A, Vázquez M, Bueno-Orovio A, Rodriguez B. In-silico human electro-mechanical ventricular modelling and simulation for drug-induced pro-arrhythmia and inotropic risk assessment. Prog Biophys Mol Biol. 2021 January; 159:58-74. doi: 10.1016/j.pbiomolbio.2020.06.007.
[30] Hansen, N., and Ostermeier, A., 1996, “Adapting Arbitrary Normal Mutation Distributions in Evolution Strategies: The Covariance Matrix Adaptation,” Proceedings of IEEE International Conference on Evolutionary Computation, Nagoya, Japan, May 20-22, pp. 312-317
[31] Muszkiewicz A, Britton O J, Gemmell P, Passini E, Sánchez C, Zhou X, Carusi A, Quinn T A, Burrage K, Bueno-Orovio A, Rodriguez B. Variability in cardiac electrophysiology: Using experimentally-calibrated populations of models to move beyond the single virtual physiological human paradigm. Prog Biophys Mol Biol. 2016 January; 120(1-3):115-27. doi: 10.1016/j.pbiomolbio.2015.12.002.
[32] Yang P C, Clancy C E. In silico Prediction of Sex-Based Differences in Human Susceptibility to Cardiac Ventricular Tachyarrhythmias. Front Physiol. 2012 Sep. 14; 3:360. doi: 10.3389/fphys.2012.00360.
[33] Fan L, Yao J, Yang C, Xu D, Tang D. Modeling Active Contraction and Relaxation of Left Ventricle Using Different Zero-load Diastole and Systole Geometries for Better Material Parameter Estimation and Stress/Strain Calculations. Mol Cell Biomech. 2016; 13(1):33-55. doi: 10.3970/mcb.2016.013.044.
[34] Trinidad F, Rubonal F, Rodriguez de Castro I, Pirzadeh I, Gerrah R, Kheradvar A, Rugonyi S. Effect of Blood Flow on Cardiac Morphogenesis and Formation of Congenital Heart Defects. J Cardiovasc Dev Dis. 2022 Sep. 8; 9(9):303. doi: 10.3390/jcdd9090303.
[35] Li Z, Dutta S, Sheng J, Tran P N, Wu W, Chang K, Mdluli T, Strauss D G, Colatsky T. Improving the In Silico Assessment of Proarrhythmia Risk by Combining hERG (Human Ether-à-go-go-Related Gene) Channel-Drug Binding Kinetics and Multichannel Pharmacology. Circ Arrhythm Electrophysiol. 2017 February; 10 (2): e004628. doi: 10.1161/CIRCEP.116.004628. Erratum in: Circ Arrhythm Electrophysiol. 2017 March; 10 (3): e000024. doi: 10.1161/HAE.0000000000000024.
[36] Tomek J, Bueno-Orovio A, Passini E, Zhou X, Minchole A, Britton O, Bartolucci C, Severi S, Shrier A, Virag L, Varro A, Rodriguez B. Development, calibration, and validation of a novel human ventricular myocyte model in health, disease, and drug block. Elife. 2019 Dec. 24; 8: e48890. doi: 10.7554/eLife.48890.
[37] Pokorney S D, Yen D C, Campbell K B, Allen LaPointe N M, Sheng S, Thomas L, Bahnson T D, Daubert J P, Picini J P, Jackson K P, Thomas K L, Al-Khatib S M. Dofetilide dose reductions and discontinuations in women compared with men. Heart Rhythm. 2018 April; 15(4):478-484. doi: 10.1016/j.hrthm.2018.01.027. PMID: 29605013.
[38] Caballero A, Mao W, Mckay R, Primiano C, Hashim S, Sun W. New insights into mitral heart valve prolapse after chordae rupture through fluid-structure interaction computational modeling. Sci Rep. 2018 Nov. 23; 8(1):17306. doi: 10.1038/s41598-018-35555-5. Erratum in: Sci Rep. 2019 Jun. 27; 9 (1): 9558. doi: 10.1038/s41598-019-44072-y.
[39] Bucelli M, Zingaro A, Africa P C, Fumagalli I, Dede' L, Quarteroni A. A mathematical model that integrates cardiac electrophysiology, mechanics, and fluid dynamics: Application to the human left heart. Int J Numer Method Biomed Eng. 2023 March; 39(3): e3678. doi: 10.1002/cnm.3678.
[40] Isotani A, Yoneda K, Iwamura T, Watanabe M, Okada J I, Washio T, Sugiura S, Hisada T, Ando K. Patient-specific heart simulation can identify non-responders to cardiac resynchronization therapy. Heart Vessels. 2020 August; 35(8):1135-1147. doi: 10.1007/s00380-020-01577-1.

In the above study results, each model described in A to C of II. METHOD is a specific example of the prediction model 341 in the above embodiment and modification. Furthermore, the personalization method described in D of II. METHOD is a specific example of processing by the identification function 354. Furthermore, the pharmacological effect prediction described in D of III. RESULTS is a specific example of processing by the prediction function 355. Furthermore, as exemplified in the personalization method of D of II. METHOD, the identification function 354 may use a function defined for each of some or all of the plurality of physical models constituting the prediction model 341 to minimize or maximize the function or to determine a parameter that minimizes or maximizes the function, thereby identifying each parameter in the some or all physical models.

According to at least one embodiment described above, the pharmacological effect for each patient can be accurately predicted.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured to
acquire subject information; and
identify parameters of at least one or more physical models among a plurality of physical models included in a prediction model for predicting a behavior of a heart using the subject information.

2. The medical information processing apparatus according to claim 1, wherein the plurality of physical models includes a first physical model and a second physical model into which an output of the first physical model is input as at least a part of an input.

3. The medical information processing apparatus according to claim 1, wherein the prediction model includes at least one physical model among a molecular dynamic model that predicts an interaction between an ion channel on a cell membrane of the heart and a drug, an electrophysiological model that predicts at least one of an electrical change and a chemical change in cells of the heart, and a cross-bridge motion model that predicts a contraction of a sarcomere in a myocardium of the heart, and a cardiac deformation model that predicts a heartbeat state of the heart based on at least one prediction result.

4. The medical information processing apparatus according to claim 3, wherein
the prediction model includes the molecular dynamic model, the electrophysiological model, the cross-bridge motion model, and the cardiac deformation model,
the electrophysiological model predicts the at least one of the electrical change and the chemical change based on a prediction result of the molecular dynamic model,
the cross-bridge motion model predicts the contraction of the sarcomere based on a prediction result of the electrophysiological model, and
the cardiac deformation model predicts the heartbeat state of the heart based on a prediction result of the cross-bridge motion model.

5. The medical information processing apparatus according to claim 3, wherein the prediction model is configured by replacing at least one of the molecular dynamic model, the electrophysiological model, the cross-bridge motion model, and the cardiac deformation model with an actual measurement value, an actual measurement formula, or another prediction value.

6. The medical information processing apparatus according to claim 3, wherein the cardiac deformation model is a deformation model that predicts deformation in a cross section of a left ventricle of the heart.

7. The medical information processing apparatus according to claim 6, wherein the deformation model predicts deformation in longitudinal cross section or a transverse cross section of the left ventricle of the heart.

8. The medical information processing apparatus according to claim 3, wherein the prediction model further includes a blood flow model that predicts blood flow of a subject based on a prediction result of the cardiac deformation model.

9. The medical information processing apparatus according to claim 1, wherein the processing circuitry is configured to acquire medical images collected in a plurality of time phases for the heart of a subject and medical information of the subject.

10. The medical information processing apparatus according to claim 9, wherein the processing circuitry is configured to acquire medical images collected for a ventricle of the heart of the subject.

11. The medical information processing apparatus according to claim 9, wherein the processing circuitry is configured to acquire the medical information including sex, blood pressure, height, and weight of the subject.

12. The medical information processing apparatus according to claim 1, wherein the processing circuitry is configured to correct a numerical value based on the acquired subject information.

13. The medical information processing apparatus according to claim 12, wherein the processing circuitry is configured to correct the numerical value based on the subject information according to a state of the subject when the subject information is obtained from the subject.

14. The medical information processing apparatus according to claim 1, wherein the processing circuitry is configured to identify the parameter of each physical model using the subject information for each of the plurality of physical models.

15. The medical information processing apparatus according to claim 14, wherein the processing circuitry is configured to identify the parameters of the respective physical models by inverse analysis using the subject information.

16. A medical information processing system comprising:
the medical information processing apparatus according to claim 1; and
a terminal apparatus that is connected to the medical information processing apparatus via a network and receives an input operation by an operator.

17. A medical information processing method comprising:
acquiring subject information; and
identifying parameters of at least one or more physical models among a plurality of physical models included in a prediction model for predicting a behavior of a heart using the subject information.

18. A storage medium non-temporarily storing a program that causes a computer to execute each of the following processes:
acquiring subject information; and
identifying parameters of at least one or more physical models among a plurality of physical models included in a prediction model for predicting a behavior of a heart using the subject information.
